(19) United States
(12) Patent Application Publication
(10) Pub. No.: US 2020/0179269 A1
(43) Pub. Date: Jun. 11, 2020

(54) VAPORIZABLE COMPOSITIONS COMPRISING CANNABINOL

(71) Applicant: KANABO RESEARCH LTD., Tel-Aviv (IL)

(72) Inventors: Avihu TAMIR, Tel Aviv (IL); Doron FRIEDMAN, Carmei-Yosef (IL); Oliver SASSON, Hadera (IL)

(21) Appl. No.: 16/495,468

(22) PCT Filed: Mar. 20, 2018
(86) PCT No.: PCT/IL2018/050318

§ 371 Date: Sep. 19, 2019

Related U.S. Application Data
(60) Provisional application No. 62/473,494, filed on Mar. 20, 2017.
(60) Provisional application No. 62/473,496, filed on Mar. 20, 2017.

(51) Int. Cl. A61K 9/00; A61K 31/352; A61K 36/185; A61P 25/20; A61K 31/01; A61K 31/05

(52) U.S. Cl. CPC A61K 9/007; A61K 31/352; A61K 31/05; A61P 25/20; A61K 31/01; A61K 36/185

(57) ABSTRACT

Vaporizable compositions based on specific components in cannabis extracts and in particular comprising cannabinol and optionally sleep inducing terpenes, are provided. Such compositions, comprising low to medium concentration of CBN, are particularly useful in treating sleep disorders such as insomnia.

### FIELD OF THE INVENTION

The present invention provides vaporizable compositions based on specific components in cannabis extracts and in particular comprising cannabinol. These compositions are useful in treating sleep disorders such as insomnia.

### BACKGROUND OF THE INVENTION

Sleep disorders are a group of conditions that affect a patient's ability to sleep well on a regular basis, thus affecting the same patient's ability to function while performing daily activities. Sleep disorders contribute to motor vehicle crashes and occupational errors, and people experiencing sleep insufficiency overtime are more likely to suffer from diseases such as hypertension, diabetes, depression, and obesity. This is a wide reaching problem, with an estimated 50-70 million US adults suffering from sleep or wakefulness disorders.

Currently available treatments of sleep disorders are unsatisfying. The most common treatment for sleep disorders is lifestyle and behavioral treatment such as relaxation training, stimulus control, or sleep restriction therapy. Medication based solutions, available on the market, are addictive and often lead to misuse.

The current evidence-based understanding of the effects of cannabis use on sleep is vague due to mixed findings from studies that typically lack appropriate rigid controls for confounding factors. Notably, medicinal cannabis use has recently been described to alleviate sleep problems based on anecdotal evidences from medicinal users. The majority of available reports refer to examining the effects of either THC and/or CBD on sleep pattern in either in vivo or in clinical studies. In addition, recently, a growing body of non-peer-reviewed evidences suggests contribution of cannabinol (CBN) to sleep promoting effects. WO 2018/23163 describes a pharmaceutical composition comprising a cannabis extract and optionally one or more pharmaceutically acceptable carriers, wherein the cannabis extract comprises a terpene fraction comprising limonene in an amount of at least about 5.4 wt % of the terpene fraction. Such composition may further comprises THC, CBD and cannabinodiol, and are useful for treating a sleep disorder.

Traditional routes of administration of cannabis, such as smoking or eating cannabis, are unsuitable for medicinal use of cannabis due to the fact that they are unreliable, difficult to dose, and unhealthy. The negative effects of the carcinogens in the smoke alone potentially outweigh the medical benefits of taking cannabis to relieve an ailment. As such, many medicinal cannabis users have turned to vaporization as a healthier and safer route of administration. Nevertheless, the myriad vaporizers available on the market today do not offer accurate and metered dosing.

New compositions capable of treating sleep disorders, and in particular, composition based on cannabis, providing a reliable dosing and safety, are required.

### SUMMARY OF THE INVENTION

In one aspect, the present invention provides a vaporizable composition, wherein the composition comprises from about 1 wt % to about 30 wt % of cannabinol (CBN) and up to about 15 wt % of at least one terpene, and wherein the composition has the viscosity in the range of about 10 cP to about 1000 cP at 60° C. In some embodiments, the composition comprises from about 3 wt % to about 25 wt % of CBN. In other embodiments, the composition comprises from about 5 wt % to about 12 wt % of terpenes. According to some embodiments, the terpenes are sleep inducing terpenes. According to another embodiment, the viscosity of the composition is in the range of from about 100 cP to about 700 cP at 60° C. According to one embodiment, the present invention provides a vaporizable composition comprising a cannabis extract, wherein the composition comprises from about 3 wt % to about 25 wt % of cannabinol (CBN) and from about 0 wt % to about 15 wt % of at least one terpene, and wherein the composition has the viscosity in the range of about 10 cP to about 1000 cP at 60° C.

The composition of the present invention may further comprise additional cannabinoids other than CBN, such as tetrahydrocannabinol (THC), cannabidiol (CBD), and cannabigerol (CBG). According to some embodiment, the composition further comprises CBD. According to one embodiment, the composition comprises from about 45 wt % to about 85 wt % of CBD. According to some embodiment, the composition further comprises THC. According to some embodiments, the composition comprises no more than 30 wt % or no more than 20 wt % of THC. According to some embodiments, the composition comprises from about 5 wt % to about 20 wt % of THC. According to other embodiments, when the CBD and THC are present, the weight ratio of CBD to THC is from 1:2 to 50:1. The cannabinoids and the terpenes of the composition of the present invention are originated from a cannabis extracts.

According to some embodiments, the compositions of the present invention comprise cannabis extract. Thus the present invention provides a composition comprising cannabis extract, wherein the composition comprises from about 1 wt % to about 30 wt % of cannabinol (CBN) and up to about 15 wt % of at least one terpene, and wherein the composition has the viscosity in the range of about 10 cP to about 1000 cP at 60° C. In some embodiments, the composition comprises from about 10 wt % to about 99 wt % of cannabis extract. In other embodiments, the composition comprises from about 15 wt % to about 60 wt % of cannabis extract. In certain embodiments, the composition comprises from about 80 wt % to about 98 wt % of cannabis extract. In certain embodiment, the extract comprises from about 80 wt % to about 98 wt % of cannabinoids and from about 2 wt % to about 20 wt % of terpenes. In other embodiments, the cannabis extract comprises from about 20 wt % to about 85 wt % of cannabinoids, and up to about 3 wt % or from about 0.1 to about 3 wt % of terpenes. According to some embodiments, all cannabinoids and/or terpenes are derived from cannabis extract(s). According to another embodiment, some of the cannabinoids and/or terpenes are derived from the cannabis extract and some are added from external sources. Such additional terpenes and/or cannabinoids may be naturally derived or synthetically manufactured.

According to some embodiments, the composition comprises only compounds naturally present in cannabis plants. In such embodiments, the composition is devoid of any excipient or additive which is not naturally present in cannabis plants. According to some embodiments, the composition comprises cannabis extract and additional cannabinoids and/or additional terpenes naturally present in cannabis plants. According to some embodiments, the composition comprises from about 5 wt % to about 25 wt % CBN, from about 2 wt % to about 12 wt % of terpenes selected from β-myrcene, linalool, eucalaptol, limonene, α-pinene, citral, linalyl acetate, borneol, nerolidol, terpenolene, and any combination thereof; wherein the viscosity of the composition is in the range of about 200 cP to about 800 cP at 60° C. According to some embodiments, such a composition comprises from 5 wt % to 15 wt % of THC.

The compositions of the present invention are useful for inducing sleep and/or for increasing the duration of sleep. Thus, the composition of the present invention is a hypnotic, sedative or sedative-hypnotic composition.

According to some embodiments, the present invention provides a composition comprising from about 3 to about 25 wt % CBN, from 50 to about 85 wt % CBD and from about 1 to about 15 wt % of β-myrcene. According to other embodiments, the composition comprises from 3 to 25 wt % CBN, from 50 to 85 wt % CBD and from about 1 to about 15 wt % of my linalool. According to some such embodiments, the composition comprises from about 10 to about 22 wt % CBN, from about 45 to about 75 wt % CBD and from about 5 to about 12 wt % of a terpene selected from linalool, β-myrcene and combination thereof. According to any one of such embodiments, the composition further comprises from about 5 to about 15 wt % THC. According to some such embodiments, the composition comprises from 10 to 22 wt % CBN, from about 45 to about 75 wt % CBD, from about 3 to about 8 wt % β-myrcene, from about 3 to about 8 wt % of linalool and from about 5 to about 15 wt % THC. According to some embodiments, such compositions synergicall increase sleep duration. According to some embodiment, the composition has a synergic hypnotic and/or synergic sedative effect. According to other embodiments, such compositions have a synergic sleep inducing effect, i.e. synergically reduce sleep onset. According to one embodiment, the composition comprises from about 60 to about 70 wt % CBD, from about 14 to about 20 wt % CBN, from about 8 to about 12 wt % THC and 10wt % β-myrcene. According to another embodiment, the composition comprises from about 60 to about 70 wt % CBD, from about 14 to about 20 wt %

CBN, from about 8 to about 12 wt % THC, about 5 wt % of β-myrcene and about 5 wt % of lonalool. According to yet another embodiment, the composition comprises from about 65 to about 75wt % CBD, from about 14 to about 20 wt % CBN, from about 2 to about 5 wt % of CBG, optionally from about 1 to about 3 wt % CBC and about 10wt % β-myrcene. According to still another embodiment, the composition comprises from about 65 to about 75wt % CBD, from about 14 to about 20 wt % CBN, from about 2 to about 5 wt % of CBG, optionally from about 1 to about 3 wt % CBC, about 5 wt % of β-myrcene and about 5 wt % of lonalool. According to some embodiments, the composition has an absorbance of less than 2 AU or less than 1 AU at 293 nm, when measured upon 1:100 dilution in ethanol.

According to some embodiments, the vaporizable composition of the present invention is formulated as a vaporizable pharmaceutical composition. According to some embodiments, the composition and the pharmaceutical composition of the present invention is for use is treating a sleep disorder in a subject in need thereof. According to some embodiments, the sleep disorder is insomnia.

According to another aspect, the present invention provides a container comprising the composition of the present invention. According to some embodiments, the container is adapted to be used, e.g. inserted, into a vaporizer. According to some embodiments, the container has a control mechanism to dose a specific volume.

According to certain aspects, the present invention provides a method for treating a sleep disorder in a subject in need thereof, said method comprising administering via vaporization the composition of the present invention. According to some embodiments, the sleep disorder is insomnia. According to other embodiment, the administration is performed using a vaporizer device. According to other embodiment, the vaporizer delivers a controlled amount of cannabinoids.

### BRIEF DESCRIPTION OF THE FIGURES

FIG. 1 shows the absorbance of the extract Comp E (Table 5) between 250 to 400 nm; the absorbance was measured upon 1:100 dilution of the extract in ethanol.

### DETAILED DESCRIPTION OF THE INVENTION

In one aspect, the present invention provides a vaporizable composition, wherein the composition comprises from about 1 wt % to about 30 wt % of cannabinol (CBN) or derivatives thereof and up to about 15 wt % of at least one terpene, wherein the composition has the viscosity in the range of about 10 cP to about 2000 cP at 60° C.

The term “cannabis” as used herein, refers to plants of the genus Cannabis, including but not limited to Cannabis sativa, Cannabis indica, and Cannabis ruderalis.

The term “extract” as used herein refers to a liquid substance obtained through extraction from a given substance. In particular, the term “extract” refers to a liquid or semi-solid or resinous substance obtained through extraction from cannabis plant. In some embodiments, the term refers to a mixture of liquid or semi-solid, resinous substances obtained through extraction from two or more different cannabis species. In some embodiments, the term refers also to a compound from purified from the extract. According to some embodiments, the term “extract” has the meaning of a mixture or combination of two or more extracts.

The term “vaporizable composition” as used herein refers to a composition that, using a suitable device, converts to a vapor that can be inhaled. In one embodiment, the vaporizable composition produces inhalable vapor upon heating e.g. using a vaporizer such as electronic cigarettes. According to some embodiments, the vaporization occurs in the temperature range of between about 120 to about 240° C. According to some embodiments, the composition of the present invention is vaporizable at the temperature range of between about 120° C. to about 250° C. According to another embodiment, the composition is vaporizable in the temperature range of between about 130° C. to about 230° C., about 140° C. to about 220 ° C., or about 150° C. to about 210° C. According to one embodiment, the composition is vaporizable in the temperature range of between about 160° C. to about 230° C. or about 170 to about 220° C. According to one embodiment, the composition is vaporizable in the temperature range of between about 180° C. to about 210° C. In one embodiment, the vaporizer delivers controlled and pre-defined volume on each puff. According to some embodiment, the vaporizable composition is of high purity and substantially devoid of waxes. According to other embodiments, the composition is substantially devoid of plant residuals such as chlorophyll. Thus, according to some embodiment, the composition liquefies at a temperature of about 35 to about 70° C. or from about 40 to about 65° C. or at a temperature between about 45 to 60° C. or from about 50 to about 55° C.

The term “cannabinoid” as used herein refers to a cannabinoid naturally present in cannabis plant or derivative of said cannabinoid. The term comprises but not limited to delta-8-tetrahydrocannabinol, delta-9-tetrahydrocannabinol, cannabidiol, cannabinol (CBN), cannabigerol, nabilone, delta-9-tetrahydro cannabinotic acid, the nonpsychotropic cannabinoid 3-dimethylnepty II carboxylic acid homologine 8, delta-8-tetrahydrocannabinol, prodrugs and pharmaceutically acceptable salts and complexes and mixtures thereof

The term “terpene” or “terpenes” are used herein interchangeably and refer to terpenes naturally present in cannabis plant. In one embodiment, the terpenes are selected from limonene, α-pinene, β-myrcene, linalool, β-caryophyllene, carophyllene oxide, nerolidol, phytol, trans-β-ocimene, α-terpinolene, trans-caryophyllene, α-humulene, linalyl acetate, borneol, caryophyllene and any combination thereof. According to another embodiment, the terpene is selected from limonene, α-pinene, β-myrcene, linalool, β-caryophyllene, carophyllene oxide, cerolidol, phytol, linalyl acetate, borneol and any combination thereof. According to one embodiment, the terpene is β-myrcene. According to another embodiment, the terpene is linalool.

The terms “cannabinol” and “CBN” are used herein interchangeably and refer to a weak psychoactive cannabinoid (CAS number 521-35-7; IUPAC name: 6,6,9-trimethyl-3-pentylbenzo[c]chromen-1-ol) having the structure presented in Formula I.

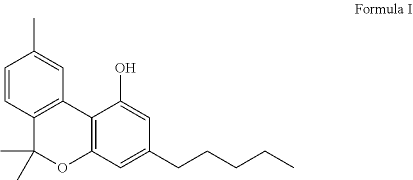 [SMILES: CCCCCc1cc(O)c2c(c1)OC(C)(C)c1ccc(C)cc1-2]

Nevertheless, in some embodiments, this term collectively refers also to all derivatives of the compound of Formula I, for example a derivative having a shortened alkyl tail, such as CBN-C4, CNB-C3 and CBN-C2. CBN is usually produced from the degradation (oxidation) of THC.

The composition of the present invention comprises from about 0.5 to 35 wt %, or from about 1 wt % to about 30 wt % of cannabinol. According to another embodiment, the composition of the present invention comprises from about 3 to about 25 wt % or from about 5 to about 20 wt % of CBN or from about 5 to about 25 wt % CBN. In other embodiments, the composition comprises from about 2 wt % to about 16 wt %, about 3 wt % to about 14 wt %, about 4 wt % to about 12 wt %, or about 6 wt % to about 10 wt % of CBN. In further embodiments, the composition comprises from about 2 wt % to about 10 wt %, or from about 4 wt % to about 8 wt %, or from about 5 to about 6 wt % of CBN. In certain embodiments, the composition comprises from about 3 wt % to about 15 wt %, about 5 wt % to about 10 wt % or about 10 to about 15 wt % of CBN. In one embodiment, the composition comprises from about 15 to about 20 wt % of CBN. In another embodiment, the composition comprises from about 15 to about 25 wt % of CBN, or from about 10 to about 25 wt %. According to yet another embodiment, the composition comprises less than about 15 wt %, or less than about 10 wt %, or less than about 8 wt % of CBN, but not less than 0.1 wt % of CBN. In other embodiments, the composition comprises more than about 1 wt %, more than about 2 wt %, or more than 4 wt % of CBN. The invention further contemplates any combination of the above embodiments. The terms “less than” and “no more than” are used herein interchangeably and refer to a value that is equal or less than the named value. Similarly, the term “up to” includes the upper value.

According to some embodiments, the composition comprises one or more terpenes in the range of about 0 wt % or about 0.1 wt % to about 15 wt %. According to other embodiments, the composition comprises at least one terpene in the range of about 1 wt % to about 15 wt %. According to some embodiments, the composition comprises about 1 to about 13 wt %, about 2 to about 12 wt %, about 3 to about 11 wt %, about 4 to about 10 wt %, about 5 to about 9 wt %, or about 6 to about 8 wt % of terpenes. According to another embodiment, the composition comprises about 1 to about 12 wt %, from about 8 to about 12 wt % or from about 5 to about 15 wt % of terpenes. According to other embodiments, the composition comprises about 2 to about 10 wt %, about 3 to about 9 wt %, about 4 to about 8 wt %, or about 5 to about 7 wt % of terpenes. According to further embodiments, the composition comprises at least 2 wt %, at least 5 wt %, at least 7 wt %, at least 10 wt % of terpenes. According to another embodiment, the composition comprises no more than about 15 wt %, or no more than about 10 wt %, of terpenes. According to another embodiment, the composition comprises no more than about 8 wt %, or no more than 5 wt %, of terpenes. According to some embodiments, the terpenes are terpenes naturally present in cannabis plant. According to one embodiment, the terpenes are sleep-inducing terpenes such as limonene, α-pinene, β-myrcene, linalool, β-caryophyllene, carophyllene oxide, nerolidol, phytol, trans-β-ocimene, α-terpinolene, trans-caryophyllene, α-humulene, linalyl acetate, borneol, caryophyllene and any combination thereof. According to another embodiment, the terpene is selected from limonene, α-pinene, β-myrcene, linalool, β-caryophyllene, carophyllene oxide, cerolidol, phytol, linalyl acetate, borneol and any combination thereof. According to one embodiment, the terpene is β-myrcene. According to another embodiment, the terpene is linalool. According to some embodiments, the composition comprises from about 1 to about 12 wt % of such terpenes, or from about 2 to about 10 wt % of such terpenes. In one embodiment, the composition comprises from about 2 to about 8 wt % of β-myrcene. In another embodiment, the composition comprises from about 2 to about 8 wt % or from about 8 to about 12 wt % of linalool.

According to any one of the above embodiments, the compositing comprises no more than 5 wt % of limonene. According to other embodiment, the composition comprises no more than 4 wt %, 3 wt %, 2 wt % or no more than 1 wt % of limonene. According to one embodiment, the composition comprises from 0 to 4, from 1 to 3 wt % of limonene. According to some embodiment, the composition of the present invention is devoid or limonene.

According to some embodiments, the terpenes are derived from a cannabis extract or synthetically manufactured. The term “0 wt %” means that the composition is devoid of that compound or comprises negligible or undetectable amount of it. Thus in some embodiments, the composition is devoid of terpenes.

According to any one of the above embodiments, the composition has a viscosity enabling use of the composition with vaporizers. According to one embodiment, the composition has a viscosity in the range of 10 cP to about 3000 cP at 60° C. The terms “centipoise” and “cP” are used herein interchangeably. According to one embodiment, the composition has a viscosity in the range of 10 cP to about 2000 cP at 60° C. According to another embodiment, the composition has a viscosity in the range of 100 cP to about 1000 cP at 60° C. According to certain embodiments, the composition has a viscosity in the range of 100 cP to about 800 cP at 60° C. or in the range of 200 cP to 800 cP. According to a further embodiment, the composition has a viscosity in the range of about 20 cP to about 950 cP, about 40 cP to about 900 cP, about 60 cP to about 850 cP, about 80 cP to about 800 cP, about 100 cP to about 750 cP, about 150 cP to about 700 cP, about 200 cP to about 650 cP, or about 300 to 700 cP at 60° C. According to another embodiment, the viscosity is in the range of 200 cP to 800 cP, 300 cP to 700 cP or 400 cP to 600 cP at 60° C. According to some embodiments, the viscosity is in the range of 100 cP to 700 cP at 60° C. According to some embodiments, the viscosity is in the range of 500 to 1000 cP, 500 to 1500 cP, 1000 to 1500 cP or 1500 to 2000 cP at 60° C.

According to some embodiments, the composition liquefies at a temperature of about 40 to about 90° C., or at a temperature of about 45 to about 80° C., or at a temperature of about 50 to about 75° C. According to other embodiments, the composition liquefies at a temperature of about 40 to about 55° C. or about 45 to about 60° C.

According to any one of the above embodiments, the composition further comprises at least one cannabinoid in addition to CBN. Thus, in one embodiment, the composition further comprises one or more cannabinoids other than CBN.

According to any one of the above embodiment, the composition comprises from about 80 wt % to about 98 wt % cannabinoids. According to some embodiments, the composition comprises from about 80 wt % to about 95 wt % cannabinoids. According to some embodiments, the composition comprises from about 82 wt % to about 96 wt %, about 84 wt % to about 94 wt %, about 86 wt % to about 92 wt %, or about 88 wt % to about 90 wt % of cannabinoids.

According to one embodiment, the cannabinoids are selected from delta-8-tetrahydrocannabinol, delta-9-tetrahydrocannabinol (THC), cannabidiol (CBD), cannabinol (CBN), cannabigerol (CBG), delta-9-tetrahydro cannabinotic acid, the nonpsychotropic cannabinoid 3-dimethylnepty 11 carboxylic acid homologine 8, delta-8-tetrahydrocannabinol. Other non-limiting examples of cannabinoids are Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM) and any combination thereof. According to one embodiment, the cannabinoids are selected from the group consisting of tetrahydrocannabinol (THC), cannabidiol (CBD), cannabigerol (CBG), and tetrahydrocannabinotic acid. According to some embodiments, the cannabinoids are derived from a cannabis extract or synthetically manufactured. According to some embodiments, the typical conversion ratio from animal model to human is 1 to 0.1 mg/kg.

According to some embodiments, the composition further comprises THC. According to one embodiment, the composition comprises no more than about 50 wt % of THC. According to other embodiments, the composition comprises no more than about 40 wt %, no more than 30 wt %, no more than about 25 wt %, no more than about 20 wt %, no more than about 15 wt %, no more than 10 wt %, no more than about 5 wt %, no more than about 4 wt %, no more than 3 wt %, no more than about 2 wt %, no more than about 1 wt %, or no more than 0.5 wt % of THC. According to certain embodiments, the composition comprises from about 5 to about 60 wt % of THC. According to other embodiments, the composition comprises from about 10 to about 55, from about 15 to about 50, from about 20 to about 45 from 25 to 40 from 30 to 35 wt % THC. According to one embodiment, the composition comprises from about 10 to about 35 wt % of THC. According to another embodiment, the composition comprises from about 40 to about 60 wt % of THC. According to one embodiment, the composition comprises from about 5 to about 20 wt % THC. According to a further embodiment, the composition comprises from 10 to about 20 wt % or from about 5 to about 15 wt % of THC. According to another embodiment, the composition comprises from 20 to 50 wt % THC. According to some embodiment, the composition comprises from 3 to 22 wt %, from about 7 to about 18 wt % or from about 8 to about 16 wt % or from about 12 to about 16 wt % of THC

According to some embodiment, the composition further comprises CBD. According to some embodiments, the composition comprises from about 10 to about 90 wt % of CBD. According to some embodiments, the composition comprises from about 10 to about 80 wt % of CBD. According to one embodiment, the composition comprises from about 10 to about 30 wt % or from about 30 to about 60 wt % or from about 60 to 85 wt % of CBD. According to another embodiment, the composition comprises from about 15 to 75 wt %, from 20 to 70 wt %, from 25 to 65 wt %, from about 30 to about 60 wt %, from about 35 to about 55 wt % of CBD. According to some embodiments, the composition comprises from about 45 to about 85 wt % or from 55 wt % to about 85 wt %, from about 60 to about 80 wt % or from about 65 to about 75 wt % of CBD. According to some embodiments, the composition comprises from about 60 to about 75 wt % of CBD. According to one embodiment, the composition comprises from 50 to 80 wt % CBD. According to yet another embodiment, the composition comprises from 40 to 90 wt % CBD or from 40 to 60 wt % CBD.

According to one embodiment, the composition comprises CBD and THC. According to some embodiments, CBD is present in a higher amount and concentration than THC. According to one embodiment, the ratio between CBD and THC (CBD:THC ratio) is at least 3 to 2. In other embodiments, the CBD:THC ratio is in the range of 2:1 to 100:1. In another embodiment, the CBD:THC ratio the in the range of 4:1 to 50:1, or in the range of 6:1 to 25:1, or in the range of 8:1 to 15:1. According to further embodiments, the CBD:THC ratio the in the range of 4:1 to 12:1 or to 10:1. According to some such embodiments, the composition comprises from about 55 to about 85 wt % CBD. According to other embodiments, the composition comprises from about 45 to about 85 wt %, or from about 50 to about 80 wt % of CBD. According to certain embodiments, the CBD:THC ratio is in the range of 2:1 to 1000:1, 4:1 to 800:1, 6:1 to 600:1, 8:1 to 400:1, or 10:1 to 200:1.

According to some embodiment, the weight ratio of terpenes to cannabinoids is in the range of about 1:50 to 1:4 or about 1:49 to about 1:10. According to another embodiment, the weight ratio of terpenes to cannabinoids is in the range of 1:30 to 1:5, 1:20 to 1:7 or 1:15 to 1:10. According to a further embodiment, the weight ratio of terpenes to cannabinoids is in the range of 1:15 to 1:5. According to some embodiments, the weight ratio of terpenes to cannabinoids is in the range 1:8 to 1:12. According to other embodiments, the weight ratio of terpenes to cannabinoids is in the range 1:15 to 1:25.

According to alternative embodiments, the CBD:THC ratio is in the range of 1:1 to 1:20, 1:2 to 1:15, 1:3 to 1:10 or 1:5 to 1:7.

According to some embodiments, the composition comprises from 5 to 25 wt % CBN and from 10 to 30 wt % CBD.

According to one embodiment, the composition comprises from 3 to 25 wt % CBN and from 50 to 85 wt % CBD. According to other embodiment, the composition comprises from 5 to 25 wt % CBN and from about 60 to about 80 wt % CBD. According to another embodiments, the composition comprises from about 10 to about 25 wt % CBN and from about 45 to about 75 wt % CBD. According to some such embodiments, the composition comprises from about 1 to about 12 wt % of a terpene selected from linalool, β-myrcene and combination thereof According to some embodiments, the composition comprises from about 1 to about 6 wt % or about 8 to about 12 wt % of such terpenes.

According to some such embodiments, the composition further comprises THC. According to some embodiments, the composition comprises from about 5 to about 20 wt % CBN, from about 10 to about 30 wt % CBD and from about 40 to about 65 wt % THC. According to another embodiment, the composition comprises from about 3 to about 15 wt %

CBN, from about 50 to about 85 wt % CBD and from about 4 to about 20 wt % THC. According to other embodiment, the composition comprises from about 5 to about 25 wt % CBN, from about 60 to about 80 wt % CBD and from about 4 to about 20 wt % THC. According to another embodiments, the composition comprises from about 10 to 25 wt % CBN, from about 45 to about 75 wt % CBD and from about 4 to about 20 wt % THC. According to some such embodiments, the composition comprises from about 3 to about 15 wt % of terpenes. According to one embodiment, the composition comprises from about 5 to about 13 wt % of terpenes. According to one embodiment, the composition comprises from about 8 to about 12 wt % or 10 wt % of terpenes. According to some embodiments, the terpene is selected from of limonene, α-pinene, β-myrcene, linalool, β-caryophyllene, carophyllene oxide, cerolidol, phytol, linalyl acetate, borneol and any combination thereof. According to one embodiment, the terpene is β-myrcene, linalool and any combination thereof. According to one embodiment, the composition comprises from about 3 to about 12 wt % of β-myrcene. According to another embodiment, the composition comprises from about 3 to about 12 wt % of linalool.

According to any one of the above embodiments, the vaporizable compositions of the present invention are useful for prolongation of sleep and for inducing sleep.

As it has been shown in the examples the compositions of the present invention comprising the CBN, CBD and terpenes such as β-myrcene or linalool provide a synergic effect on a speed of falling asleep and on the duration of sleep. According to any one of the above embodiments, the composition of the present invention has a synergic effect of promoting sleep induction (sleep onset). According to any one of the above embodiments, the composition of the present invention has a synergic effect of increasing a duration of sleep. According to some embodiments, the composition has a synergic hypnotic effect. According to other embodiments, the composition has a synergic sedative effect.

According to some embodiments, the composition further comprises at least one cannabinoid selected from CBG, CBC, CBV, THCA, cannabidiolic acid (CBDA) and any combination thereof. According to some embodiments, the content of said at least one cannabinoid is from about 0.5 to about 10 wt %. According to some embodiment, the content of said at least one cannabinoid is from about 1 wt % to about 8 wt %, from about 2 wt % to about 6 wt %, or from 3 to 5 wt %.

According to another embodiment, the composition comprises CBG, CBC, CBV, THCA and CBDA. According to some embodiments, the total content of said cannabinoids is from about 2 to about 10 wt % or from about 3 to about 8 wt %. According to one embodiments, the total content of said cannabinoids is from about 2 to about 8 wt %, from about 3 to about 6 wt %.

According to some embodiments, the composition further comprises residual solvents and ash matter. According to one embodiment, the residual solvents are hydrophobic solvents. According to another embodiment, the residual solvents are selected from ethanol, butane, propane, isopropyl alcohol, ethyl acetate, cyclohexane, butanol, hexylene glycol.

According to any one of the above embodiments, the composition of the present invention is useful for increasing the duration of sleep. The composition of any one of the above embodiment is also useful for decreasing the sleep onset. Thus the composition of the present invention is a sleep prolonging or a hypnotic and/or a sedative composition or sleep promoting composition. Thus the term “composition” may be substituted by the term “hypnotic composition”, by the term “sedative composition” or by term “sedative-hypnotic composition”. Thus, the present invention provides a hypnotic vaporizable composition, wherein the composition comprises from about 1 wt % to about 30 wt % of cannabinol (CBN) or derivatives thereof and up to about 15 wt % of at least one terpene, wherein the composition has the viscosity in the range of about 10 cP to about 2000 cP at 60° C. According to another embodiment, the present invention provides a sedative vaporizable composition, wherein the composition comprises from about 1 wt % to about 30 wt % of cannabinol (CBN) or derivatives thereof and up to about 15 wt % of at least one terpene, wherein the composition has the viscosity in the range of about 10 cP to about 2000 cP at 60° C. Any other limitations and embodiments of the present invention are valid for such compositions. The term “hypnotic” as used herein refers to a composition used or capable of inducing or maintaining a sedation or sleep. The term “sedative” as used herein has the meaning of calming and inducing sleep. The term “sleep onset” as used herein refers to a time required for the transition from wakefulness into sleep. According to some embodiments, increased or synergic hypnotic effect comprises (i) increased or synergic effect on sleep onset, (ii) increased or synergic hypnotic effect on duration of sleep, or (iii) both (i) and (ii).

According to some embodiments, the present invention provides a vaporizable composition comprising from 3 to 25 wt % CBN, from 50 to 85 wt % CBD and from 1 to 15 wt % of β-myrcene. According to other embodiments, the composition comprises from 3 to 25 wt % CBN, from 50 to 85 wt % CBD and from 1 to 15 wt % of my linalool. According to some such embodiments, the composition comprises from 10 to 22 wt % CBN, from 45 to 75 wt % CBD and from 5 to 12 wt % of a terpene selected from linalool, β-myrcene and combination thereof. According to such embodiments, the composition further comprises from 5 to 15 wt % THC. According to some such embodiments, the composition comprises from 10 to 22 wt % CBN, from 45 to 75 wt % CBD, from 3 to 8 wt % β-myrcene, from 3 to 8 wt % of linalool and from 5 to 15 wt % THC. According to some embodiments, such compositions have a synergic sleep inducing effect. According to other embodiments, such compositions have a synergic effect of increasing sleep duration. According to some embodiments, the composition has a synergic hypnotic effect. According to other embodiments, the composition has a synergic sedative effect. According to one embodiment, the composition comprises from 60 to 70 wt % CBD, from 14 to 20 wt % CBN, from 8 to 12 wt % THC and (i) 10 wt % β-myrcene or (ii) 5 wt % of β-myrcene and 5 wt % of lonalool. According to another embodiment, the composition comprises from 65 to 75 wt % CBD, from 14 to 20 wt % CBN, from 2 to 5 wt % of CBG, optionally from 1 to 3 wt % CBC and (i) 10 wt % β-myrcene or (ii) 5 wt % of β-myrcene and 5 wt % of lonalool. According to some embodiment, the composition further comprises CBG, CBC, CBV, THCA and CBDA, wherein the total content of said cannabinoids is from about 2 to 8 wt %. According to some embodiments, the composition further comprises at least one terpene selected from eucalaptol, limonene, α-pinene, citral, linalyl acetate, and any combination thereof. According to one such embodiment, the composition comprises from 3 to 6 wt % of β-myrcene.

According to some embodiments, the composition comprises from 3 to 25 wt % CBN, from 50 to 85 wt % CBD, from 5 to 15 wt % THC and from 1 to 10 wt % of 13-myrcene. According to certain embodiments, the composition comprises from 10 to 20 wt % CBN, from 55 to 75 wt % CBD, from 5 to 15 wt % THC and from 8 to 12 wt % of β-myrcene. According to other embodiments, the composition comprises from 3 to 25 wt % CBN, from 50 to 85 wt % CBD, from 5 to 15 wt % THC and from 1 to 10 wt % of my linalool. According to some such embodiments, the composition comprises from 10 to 15 wt % CBN, from 45 to 65 wt % CBD and from 2 to 8 wt % of a terpene selected from linalool, β-myrcene and combination thereof. According to other embodiments, such compositions have a synergic sleep prolonging effect. According to some embodiments, the composition has a synergic hypnotic effect. According to other embodiments, the composition has a synergic sedative effect.

According to some embodiments, the composition of the present invention has a content as described in Tables 1 and 2 below. Each composition in Table 1 and 2 represents a separate embodiment of the invention.

TABLE 1
Compositions of the present invention

| Composition | A1 | A2 | A3 | A4 | A5 | A6 | A7 | A8 | A9 | A10 | A11 |
|---|---|---|---|---|---|---|---|---|---|---|---|
| Ingredients | wt % | wt % | wt % | wt % | wt % | wt % | wt % | wt % | wt % | wt % | wt % |
| Pure CBD 98% | 73 | 65 | 67 | 80 | 73 | 65 | 67 | 80 | 65 | 72 | 72 |
| Pure CBN 98% | 7 | 14 | 20 | 0 | 7 | 15 | 20 | 0 | 19 | 12 | 11 |
| Pure THC 90% | 10 | 11 | 0 | 0 | 10 | 10 | 0 | 0 | 10 | 0 | 0 |
| Pure CBG 98% | 0 | 0 | 3 | 10 | 0 | 0 | 3 | 10 | 0 | 4 | 5 |
| Pure CBC 99% | 0 | 0 | 0 | 0 | 0 | 0 | 0 | 0 | 0 | 2 | 2 |
| Linalool | 0 | 0 | 0 | 0 | 10 | 10 | 10 | 10 | 3 | 5 | 5 |
| Myrcene | 10 | 10 | 10 | 10 | 0 | 0 | 0 | 0 | 3 | 5 | 5 |
| Total | 100 | 100 | 100 | 100 | 100 | 100 | 100 | 100 | 100 | 100 | 100 |

TABLE 2
Compositions of the present invention

| Composition | A12 | A13 | A14 | A15 | A16 | A17 | A18 | A19 | A20 | A21 | A22 |
|---|---|---|---|---|---|---|---|---|---|---|---|
| Ingredients | wt % | wt % | wt % | wt % | wt % | wt % | wt % | wt % | wt % | wt % | wt % |
| Pure CBD 98% | 79 | 62 | 66 | 65 | 67 | 79 | 62 | 66 | 65 | 67 | 65 |
| Pure CBN 98% | 5 | 20 | 15 | 12 | 5 | 10 | 20 | 15 | 12 | 5 | 14 |
| Pure THC 90% | 0 | 6 | 7 | 10 | 15 | 0 | 6 | 7 | 10 | 15 | 11 |
| Pure CBG 98% | 6 | 2 | 0 | 0 | 3 | 6 | 2 | 0 | 0 | 3 | |
| Pure CBC 99% | 0 | 0 | 2 | 3 | 0 | 0 | 0 | 2 | 5 | 0 | 0 |
| Linalool | 3 | 7 | 5 | 2 | 2 | 1 | 0 | 4 | 0 | 0 | 0 |
| Myrcene | 3 | 1 | 0 | 4 | 2 | 0 | 0 | 0 | 0 | 0 | 10 |
| Borneol | 2 | 1 | 0 | 1 | 2 | 2 | 0 | 1 | 4 | 0 | 0 |
| Nerolidol | 2 | 0 | 3 | 2 | 2 | 2 | 7 | 2 | 4 | 10 | 0 |
| Terpinolene | 0 | 1 | 2 | 1 | 2 | 0 | 3 | 3 | 0 | 0 | 0 |
| Total | 100 | 100 | 100 | 100 | 100 | 100 | 100 | 100 | 100 | 100 | 100 |

According to any one of the above embodiments, the composition comprises cannabis extract. According to certain embodiments, the composition comprises from about 10 wt % to about 99 wt % of cannabis extract. According to some embodiments, the composition comprises from about 80 wt % to about 99 wt % or about 80 wt % to about 98 wt % of cannabis extract. According to one embodiment, the composition comprises from about 82 wt % to about 98 wt %, about 85 wt % to about 95 wt % or from about 87 wt % to about 92 wt % of cannabis extract. In one embodiment, the composition comprises about 85 wt % to about 98 wt % of cannabis extract or about 90 wt % to about 95 wt % of the extract. According to another embodiment, the composition comprises about 90 wt % to about 99 wt %, about 92 wt % to about 98 wt % or about 94 wt % to about 96 wt % of cannabis extract. According to other embodiments, the composition comprises from 30 to 70 wt % of cannabis extract. According to some embodiments, the composition comprises from 35 to 65 wt % or from about 40 to about 60 wt % of cannabis extract. According to some embodiments, the composition comprises from 40 to 55 wt % of cannabis extract. According to yet another embodiment, the composition comprises from 10 to 55 wt % of cannabis extract. According to another embodiment, the composition comprises from 15 to 50 wt %, from about 20 to about 45 wt %, from 25 to 40 wt % or from 30 to 35 wt % of cannabis extract.

According to some embodiments, the cannabis extract comprises from about 80 wt % to about 98 wt % of cannabinoids. According to other embodiment, the cannabis extract comprises from about 2 wt % to about 20 wt % of one or more terpenes. According to some embodiments, the cannabis extract comprises from about 80 wt % to about 98 wt % of cannabinoids and from about 2 wt % to about 20 wt % of one or more terpenes. According to one embodiment, the extract comprises from about 82 to 98 wt %, about 85 to about 95 wt % or from about 87 wt % to about 92 wt % of cannabinoids. According to some embodiments, the extract comprises about 90 wt % to about 98 wt %, about 92 wt % to about 98 wt % or about 94 wt % to about 96 wt % of cannabinoids. According to another embodiment, the extract comprises at least 85 wt %, at least 90 wt %, at least 92 wt % or at least 94 wt % of cannabinoids. According to such embodiments, the extract comprises from about 2 to 20 wt %, about 4 to about 18 wt %, about 8 to about 16, about 6 to about 14, or about 8 to about 12 wt % of terpenes. According to further such embodiment, the extract comprises about 1 wt % to about 10 wt %, about 2 wt % to about 9 wt %, about 3 wt % to about 8 wt %, about 4 wt % to about 7 wt %, or about 5 wt % to about 6 wt % of terpenes. According to some embodiments, the extract comprises from 5 to 40 wt % of cannabinoids. According to one embodiment, the extract comprises from about 90 wt % to about 96 wt % of cannabinoids and from about 4 wt % to about 10 wt % of terpenes. According to another embodiment, the extract comprises from about 10 to about 35 wt % or from about 15 to about 30 wt % or from about 20 to about 25 wt % or cannabinoids. According to certain embodiments, the extract comprises from about 20 to about 90 wt % of cannabinoids. According to one embodiment, the extract comprises from about 25 to about 85 wt %, from about 30 to about to about 80 wt % of cannabinoids. According to yet another embodiment, the extract comprises from about 35 to about 75 wt % or from about 40 to about 70 wt % or from about 45 to about 65 wt % of cannabinoids. According to one embodiment, the extract comprises from about 10 to about 23 wt % of cannabinoids such as THC and CBD. According to some embodiments, the extract comprises up to 5, up to 4 up to 3 or up to 2 wt % of terpenes. According to one embodiment, the extract comprises from 1 to 4 wt % or from about 2 to about 3 wt % of terpenes. According to some embodiment, the extract comprises from about 6 to about 60 wt % of cannabinoids and up to 3 wt % terpenes. According to one embodiment, the extract comprises from about 10 to about 25 wt % of cannabinoids and up to 2 wt % terpenes. According to another embodiment, the extract comprises from about 25 to about 85 wt % of cannabinoids and up to 2 wt % terpenes. According to yet another embodiment, the extract comprises from about 30 to about 80 wt % of cannabinoids and up to 2 wt % terpenes.

According to some embodiments, the extract is a commercially available extract. According to one embodiment, the extract is selected from Avidekel™ BHO extract comprising about 4 wt % THC, about 72 wt % CBD and about 2 wt % CBN, Tachllta Till™ BHO extract comprising about 76 wt % THC and about 2 wt % CBD, Blue Sky™ BHO extract comprising about 32 wt % of THC and about 0.4 wt % of CBD and Jerusalem Espress™ BHO extract comprising about 54 wt % THC about 2 wt % CBD and about 1 wt % CBN. According to one embodiment, the compositing comprises from about 10 to about 20 wt % of Tachllta Till™ BHO extract, from about 10 to about 20 wt % of Blue Sky™ BHO extract, from about 10 to about 20 wt % of Jerusalem Espress™ BHO, from about 35 to about 50 wt % of additional CBD, about 5 to about 10 wt % of additional CBN, about 1 to about 3 wt % of linalool and about 3 to about 8 wt % of β-myrcene.

According to some embodiments, the composition comprises only compounds that are naturally present in cannabis plants. According to one embodiment, all the terpenes and cannabinoids of the composition originate from the cannabis extract. According to another embodiment, all the terpenes originate from the cannabis extract and the cannabinoids originate from the cannabis extract and from additional cannabinoids. According to a further embodiment, all the cannabinoids originate from the cannabis extract and the terpenes originate from the cannabis extract and from additional terpenes. According to yet another embodiment, all or some of the cannabinoids originate from the cannabis extract and all the terpenes are additional terpenes.

The term “additional terpenes” refers to terpenes naturally present in cannabis plant and artificially added to the composition. In some embodiments, the terms “terpenes” and “additional terpenes” may be used interchangeably and all limitations and definitions related to terpenes hold for additional terpenes.

The term “additional cannabinoids” refers to cannabinoids naturally present in cannabis plant and artificially added to the composition. In some embodiments, the terms “cannabinoids” and “additional cannabinoids” may be used interchangeably and all limitations and definitions related to terpenes hold for additional cannabinoids.

According to some embodiments, all the terpenes are originated from the extract.

According to another embodiment, the terpenes of the composition are originated from the extract and from terpenes further added to the composition. According to an alternative embodiment, all the terpenes are originated from the additionally added terpenes.

According to some embodiments, all the cannabinoids are originated from the extract. According to another embodiment, the cannabinoids of the composition are originated from the extract and from cannabinoids further added to the composition. According to an alternative embodiment, all the cannabinoids are originated from the additionally added cannabinoids.

According to some embodiments, the composition comprises cannabis extract and additional cannabinoids and additional terpenes. According to one embodiment, the composition comprises from 20 to 55 wt % of additional CBD, from 3 to 25 wt % additional CBN, and from 1 to 5 wt % of additional terpenes. According to one embodiment, the terpene is selected from linalool, β-myrcene and combination thereof. According to one embodiment, the composition comprises from 25 to 50 wt % from 30 to 45 wt % or from 35 to 40 wt % of additional CBD. According to another embodiment, the composition comprises from about 5 to about 20 wt % or from about 10 to about 15 wt % of additional CBN.

According to some embodiments, the composition of the present invention has a content as described in Tables 3 and 4 below. Each composition in the tables represent a separate embodiment of the invention.

TABLE 3
Compositions of the present invention comprising cannabis extract(s)

| Composition | B1 | B2 | B3 | B4 | B5 | B6 | B7 | B8 | B9 | B10 | B11 |
|---|---|---|---|---|---|---|---|---|---|---|---|
| Ingredients | wt % | wt % | wt % | wt % | wt % | wt % | wt % | wt % | wt % | wt % | wt % |
| Avidekel ™ BHO | 42 | 0 | 0 | 0 | 0 | 0 | 0 | 0 | 10 | 20 | 0 |
| extract | | | | | | | | | | | |
| Tachllta Till ™ BHO | 0 | 30 | 0 | 0 | 15 | 15 | 15 | 5 | 5 | 5 | 10 |
| extract | | | | | | | | | | | |
| Blue Sky ™ BHO | 0 | 0 | 33 | 0 | 15 | 15 | 15 | 10 | 10 | 10 | 10 |
| extract | | | | | | | | | | | |
| Jerusalem Espress ™ | 0 | 0 | 0 | 50 | 15 | 15 | 15 | 20 | 20 | 20 | 5 |
| BHO extract | | | | | | | | | | | |
| Pure CBD 99% | 32 | 49 | 49 | 38 | 38 | 44 | 43 | 51 | 46 | 26 | 58 |
| Pure CBN 99% | 20 | 14 | 8 | 4 | 11 | 5 | 7 | 9 | 5 | 15 | 11 |
| Linalool | 3 | 2 | 5 | 8 | 1 | 3 | 1 | 4 | 2 | 2 | 1 |
| Myrcene | 3 | 5 | 5 | 0 | 5 | 3 | 4 | 1 | 2 | 2 | 5 |
| Total | 100 | 100 | 100 | 100 | 100 | 100 | 100 | 100 | 100 | 100 | 100 |

TABLE 4
Compositions of the present invention comprising cannabis extract(s)

| Composition | B12 | B13 | B14 | B15 | B16 | B17 | B18 | B19 | B20 | B21 | B22 |
|---|---|---|---|---|---|---|---|---|---|---|---|
| Ingredients | wt % | wt % | wt % | wt % | wt % | wt % | wt % | wt % | wt % | wt % | wt % |
| Avidekel ™ BHO | 0 | 0 | 0 | 10 | 20 | 0 | 0 | 0 | 10 | 20 | 0 |
| extract | | | | | | | | | | | |
| Tachllta Till ™ BHO | 10 | 10 | 5 | 5 | 5 | 10 | 10 | 5 | 5 | 5 | 15 |
| extract | | | | | | | | | | | |
| Blue Sky ™ BHO | 9 | 8 | 0 | 10 | 10 | 14 | 8 | 7 | 15 | 20 | 15 |
| extract | | | | | | | | | | | |
| Jerusalem Espress ™ | 5 | 0 | 10 | 16 | 20 | 5 | 5 | 10 | 16 | 20 | 15 |
| BHO extract | | | | | | | | | | | |
| Pure CBD 99% | 65 | 70 | 70 | 50 | 30 | 65 | 70 | 70 | 50 | 30 | 43 |
| Pure CBN 99% | 5 | 7 | 10 | 5 | 11 | 0 | 2 | 3 | 0 | 1 | 7 |
| Linalool | 3 | 1 | 4 | 2 | 2 | 3 | 1 | 4 | 2 | 2 | 1 |
| Myrcene | 3 | 4 | 1 | 2 | 2 | 3 | 4 | 1 | 2 | 2 | 4 |
| Total | 100 | 100 | 100 | 100 | 100 | 100 | 100 | 100 | 100 | 100 | 100 |

According to any one of the above embodiments, the compositions are hypnotic, sedative or sedative-hypnotic compositions.

According to one embodiment, the composition comprising exclusively compounds naturally present in cannabis plants is devoid of any excipient, compound or additive which is not present in the cannabis plant. The terms “substantially devoid”, “essentially devoid”, “devoid”, “does not include” and “does not comprise” may be used interchangeably and refer to composition that does not include, contain or comprise a particular compound, e.g., said composition comprises less than 0.1 wt %, less than 0.01 wt %, or less than 0.001 wt % of the compound. In some embodiments, the term “devoid” contemplate compositions devoid of the compound except for residual traces of the compound that might been remained for example as a result of processing such as a purification process. The term “additive” as used herein refers to any compound added to a composition, which is not naturally present in cannabis plant. It should be understood that the term devoid may comprise composition in which traces of compounds used in the process of purification are present. Nevertheless, the amount of such compounds is neglectable, e.g. the amount of such compound is less than 0.001 wt % or less than 0.0001 wt % of the composition. Thus, according to some embodiments, the composition is devoid of additives. According to some embodiments, the additives are selected from the group consisting of oils, waxes, propylene glycol, butylene glycol, hexylene glycol, glycerin, polyethylene glycol, organic solvent, vitamin E and any combination thereof. According to some embodiment, the oils are selected from natural oil and synthetic oil. Such oils exclude oils naturally present in cannabis plants. According to another embodiment, glycerin is a glycerol derived from plant oils commonly referred to as vegetable glycerin. According for yet another embodiment, organic solvent is selected from ethanol, propanol, butanol, and isopropanol.

According to any one of the above embodiments, the composition has a peak of absorbance at 293 nm. According to some embodiments, the absorbance is measured upon dilution of the composition in ethanol. According to some embodiments, the absorbance of the composition at 293 nm is less than 3 absorbance units (AU), less than 2 AU, less than 1 AU or less than about 0.5 AU, when measured upon 1 to 100 dilution of the composition in ethanol.

According to some embodiments, the composition of the present invention comprises solely compounds naturally present in cannabis plants, wherein the composition comprises cannabis extract, and optionally additional cannabinoids and/or additional terpenes, wherein the composition comprises from about 1 wt % to about 30 wt % of cannabinol (CBN) and up to about 15 wt % of terpenes, and has the viscosity in the range of about 10 cP to about 1000 cP at 60° C. According to one embodiment, the composition comprises from about 85 wt % to about 99 wt % or to about 98 wt % of cannabis extract or from about 15 to about 60 wt % of cannabis extract. According to another embodiment, such composition comprises one or more additional terpene. According to another embodiment, the composition comprises from about 85 wt % to about 99 wt % of cannabis extract and additional cannabinoid, for example selected from CBN, CBD and combination thereof. According to another embodiment, the composition comprises from about 15 to about 60 wt % of cannabis extract and additional cannabinoid, for example selected from CBN, CBD and combination thereof. According to yet another embodiment, the composition comprises from about 85 wt % to about 99 wt % of cannabis extract, additional terpenes and additional cannabinoid. According to yet another embodiment, the composition comprises from about 15 to about 60 wt % of cannabis extract, additional terpenes and additional cannabinoid. In one embodiment, the composition comprises from about 3wt % to about 25 wt % CBN, and from about 2 wt % to about 12 wt % of terpene, e.g. selected from α-pinene, β-myrcene, linalool, β-caryophyllene, carophyllene oxide, cerolidol, phytol, linalyl acetate, borneol, and combination thereof. In other embodiments, the viscosity of the composition is in the range of about 100 cP to about 700 cP at 60° C. According to some embodiments, the composition comprises less than 25 wt %, less than 15 wt %, less than 5 wt %, less than 2 wt %, or less than 1 wt % of THC. According to other embodiments, the composition comprises at least 40 wt %, at least 50 wt %, at least 60 wt % or at least 70 wt % CBD. According to some embodiments, the composition has an absorbance of less than 2 AU or less than 1 AU at 293 nm, when measured upon 1:100 dilution in ethanol.

In any one of the above embodiments, the term comprising has the meaning of consisting, thus in some embodiments, the present invention provides a vaporizable composition consisting of cannabis extract, e.g., from about 85 to about 99 wt % or to about 98 wt % of cannabis extract and optionally additional cannabinoids and/or additional terpenes naturally present in cannabis plants, wherein the composition comprises from about 1 wt % to about 30 wt % of cannabinol (CBN) and from about 0 wt % to about 15 wt % of at least one terpene, and has the viscosity in the range of about 100 cP to about 1000 cP at 60° C. According to other embodiments, the present invention provides a vaporizable composition consisting of cannabis extract, e.g., from about 15 to about 60 wt % of cannabis extract and optionally additional cannabinoids and/or additional terpenes naturally present in cannabis plants, wherein the composition comprises from about 1 wt % to about 30 wt % of cannabinol (CBN) and from about 0 wt % to about 15 wt % at least one terpenes, and has the viscosity in the range of about 100 cP to about 1000 cP at 60° C.

According to one embodiment, the composition consists of about 85 wt % to about 99 wt % cannabis extract and additional terpenes. According to another embodiment, the composition consists of about 85 wt % to about 99 wt % cannabis extract and additional cannabinoid, for example selected from CBN, CBD and combination thereof. According to yet another embodiment, the composition consists of about 85 wt % to about 99 wt % cannabis extract, additional terpenes and additional cannabinoid. According to one embodiment, the composition consists of about 15 to about 60 wt % cannabis extract and additional terpenes. According to another embodiment, the composition consists of about 15 to about 60 wt % cannabis extract and additional cannabinoid, for example selected from CBN, CBD and combination thereof. According to yet another embodiment, the composition consists of about 15 to about 60 wt % cannabis extract, additional terpenes and additional cannabinoid. According to some such embodiments, the composition comprises from 3 to 25 wt % CBN, from 50 to 85 wt % CBD and from 1 to 15 wt % of a terpene, e.g. selected from limonene, α-pinene, β-myrcene, linalool, β-caryophyllene, carophyllene oxide, cerolidol, phytol, linalyl acetate, bomeol, and combination thereof. According to some embodiments, the viscosity of the composition is in the range of from about 200 cP to about 800 cP at 60° C. According to other embodiments, the viscosity of the composition is in the range of from about 100 cP to about 700 cP or about 400 to about 600 at 60° C. According to such some embodiments, the composition further comprises from 5 to 15 wt % THC. According to some embodiments, the composition has an absorbance of less than 2 AU or less than 1 AU at 293 nm, when measured upon 1:100 dilution in ethanol.

According to some alternative embodiments, the composition comprise one or more additives. According to one embodiment, the composition comprises an additive selected from the group consisting of an oil, triglyceride, wax, propylene glycol, glycerin, butylene glycol, hexylene glycol, polyethylene glycol, organic solvent and vitamin E. According to some embodiments, the additive is selected from a triglyceride oil, capric triglyceride, caprylic triglyceride, tocopherol, tocopherol succinate or derivatives or salts thereof

According to any one of the above embodiment, the composition is suitable for use with a container or cartridge configured to be used or inserted into any vaporizer device. The term “vaporizer” as used herein refers to a device used to vaporize a composition, in particular the composition of the present invention. Example of such vaporizer are cartomizers, atomizers and clearomizer. According to some embodiments, the vaporizer utilizes a wick and coil heating element or a heating chamber such as ceramic heating chamber or titanium/quartz heating chamber. According to some embodiment, the container, cartridge or tank containing the composition is a disposable or refillable container, cartridge or tank. According to some embodiments, the vaporizer is an extraction vaporizer.

According to any one of the above embodiments, the composition is suitable for use with a container or cartridge configuration and vaporizer or e-cigarette that has a mechanism of control of volume of the vapor.

According to any one of the above embodiments, the composition formulated as a pharmaceutical composition. Thus the term composition used in any one of the above embodiments encompasses the term pharmaceutical composition and may be replaced by it. The term “pharmaceutical composition” as used herein refers to a composition comprising the composition of the present invention formulated together with one or more pharmaceutically acceptable carriers. The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. The compositions may comprise solid carriers or excipients such as, for example, lactose, starch or talcum or liquid carriers such as, for example, water, fatty oils or liquid paraffins.

Thus in one embodiment, the present invention provides a vaporizable pharmaceutical composition comprising from about 1 wt % to about 30 wt % of cannabinol (CBN) and up to about 15 wt % of at least one terpene, and wherein the composition has the viscosity in the range of about 10 cP to about 1000 cP at 60° C. According to some embodiments, the pharmaceutical composition comprises from 3 to 25 wt % CBN, from 50 to 85 wt % CBD and from 1 to 15 wt % of a terpene, e.g. selected from limonene, α-pinene, β-myrcene, linalool, β-caryophyllene, carophyllene oxide, cerolidol, phytol, linalyl acetate, borneol, and combination thereof. According to some embodiments, the pharmaceutical composition comprises from 3 to 25 wt % CBN, from 50 to 85 wt % CBD and from 1 to 15 wt % of β-myrcene. According to other embodiments, the pharmaceutical composition comprises from 3 to 25 wt % CBN, from 50 to 85 wt % CBD and from 1 to 15 wt % of my linalool. According to some such embodiments, the pharmaceutical composition comprises from 10 to 22 wt % CBN, from 45 to 75 wt % CBD and from 5 to 12 wt % of a terpene selected from linalool, β-myrcene and combination thereof. According to such embodiments, the pharmaceutical composition further comprises from 5 to 15 wt % THC. According to some such embodiments, the pharmaceutical composition comprises from 10 to 22 wt % CBN, from 45 to 75 wt % CBD, from 3 to 8 wt % β-myrcene, from 3 to 8 wt % of linalool and from 5 to 15 wt % THC. According to some embodiments, such pharmaceutical compositions have a synergic effect of increasing sleep duration. According to other embodiments, such pharmaceutical compositions have a synergic sleep inducing effect. According to some embodiments, the composition has a synergic hypnotic effect. According to other embodiments, the composition has a synergic sedative effect. According to one embodiment, the pharmaceutical composition comprises from 60 to 70wt % CBD, from 14 to 20 wt % CBN, from 8 to 12 wt % THC and (i) 10wt % β-myrcene or (ii) 5 wt % of β-myrcene and 5 wt % of lonalool. According to another embodiment, the composition comprises from 65 to 75wt % CBD, from 14 to 20 wt % CBN, from 2 to 5 wt % of CBG, optionally from 1 to 3 wt % CBC and (i) 10 wt % β-myrcene or (ii) 5 wt % of β-myrcene and 5 wt % of lonalool. According to some embodiments, the pharmaceutical composition has an absorbance of less than 2 AU or less than 1 AU at 293 nm, when measured upon 1:100 dilution in ethanol.

According to some embodiments, such pharmaceutical composition comprises from about 90 wt % to about 99 wt %, about 92 wt % to about 98 wt % or about 94 wt % to about 96 wt % of cannabis extract. According to other embodiments, the composition comprises from about 30 to about 70 wt % of cannabis extract. According to some embodiments, the composition comprises from about 35 to about 65 wt % or from about 40 to about 60 wt % of cannabis extract. According to some embodiments, the composition comprises from about 40 to about 55 wt % of cannabis extract. According to yet another embodiment, the composition comprises from about 10 to about 55 wt % of cannabis extract. According to another embodiment, the composition comprises from about 15 to about 50 wt %, from about 20 to about 45 wt %, from about 25 to about 40 wt % or from 30 to 35 wt % of cannabis extract. According to some embodiments, the composition comprising cannabis extract further comprises additional cannabinoids and additional terpenes. According to one embodiment, the composition comprises from 20 to 55 wt % of additional CBD, from 3 to 25 wt % additional CBN, and from 1 to 5 wt % of additional terpenes. According to one embodiment, the terpene is selected from linalool, β-myrcene and combination thereof. According to one embodiment, the composition comprises from 25 to 50 wt % from 30 to 45 wt % or from 35 to 40 wt % of additional CBD. According to another embodiment, the composition comprises from about 5 to about 20 or from 10 to 15 wt % of additional CBN.

The term “composition” encompassed also the term “hypnotic composition”, “sedative compositing” and “sedative-hypnotic composition”. Thus, in some embodiments, the present invention provides a hypnotic vaporizable pharmaceutical composition comprising from about 1 wt % to about 30 wt % of cannabinol (CBN) and up to about 15 wt % of at least one terpene, and wherein the composition has the viscosity in the range of about 10 cP to about 1000 cP at 60° C.

According to any one of the above embodiments, the composition or the pharmaceutic composition of the present invention is for use in treating a sleep disorder. According to some embodiments, the sleep disorder is insomnia. According to another embodiment, the composition is administered via vaporizing. According to other embodiments, the composition or the pharmaceutic composition of the present invention is for use in for inducing sleep in a subject. According to certain embodiments, the composition or the pharmaceutic composition of the present invention is for use in for increasing sleep duration in a subject. In further embodiment, administration is performed via a vaporizer device. In some embodiment, treating comprises administering the composition such that the administered dose of CBN is in the range of about 1 to about 150 mg/day. According to some embodiments, treating comprises administering about 10 mg/ day to 140 mg/day, about 20 mg/day to about 120 mg/day, about 30 mg/day to about 100 mg/day, about 40 mg/day to about 80 mg/day or about 50 mg/day to about 60 mg/day. According to one embodiment, treating comprises administering of from 1 to 40 mg/day, from 40 to 80 mg/day or from 80 to 120 mg/day. According to some embodiments, treating comprises administering of about 1.5 to 30 mg/day, about 2 to 25 mg/day, about 3 to about 20 mg/day, about 4 to about 15 mg/day or about 5 to about 10 mg/day of CBN. According to other embodiments, the treating comprises administering of about 1 to about 10 mg/day, about 2 to about 8 mg/day, or about 4 to about 6 mg/day of CBN. According to some embodiments, the treating comprises administering of about 5 to 20 mg/day or about 10 to 15 mg/day of CBN. According to certain embodiments, the treating comprises administering from 10 to about 20 mg/day of CBN. Administration to some embodiments, administering is performed in one dose or in separate doses, e.g. administering in 2, 3, 4 or more puffs. According to some embodiments, treating comprises administering 1, 2, 3 or 5 mg of CBN 1, 2, 3 or 4 times a day. According to other embodiments, treating comprises administering 10 mg of CBN 1, 2, 3 or 4 times a day. According to other embodiments, treating comprises administering 40 mg one a day, or administering 15, 20 or 30 mg 2 times a day or administering 40 mg 3 times a day. According to one embodiment, treating comprises administering of about 1 to about 10 mg of the composition of the present invention 1, 2, 3 or 4 times a day. According to another embodiment, treating comprises administering of about 2 to about 8 mg of the composition of the present invention 1, 2, 3 or 4 times a day. According to still another embodiment, treating comprises administering of about 3 to about 6 mg of the composition of the present invention 1, 2, 3 or 4 times a day. According to some embodiments, the dose is composed from one or more puffs. According to some embodiments, each dose comprises from 1 to 10 puffs, e.g. 1, 2, 3, 4, or 5 puffs. According to some embodiments, the puffs are measured puffs, i.e. each puff releases measured amount of the composition of the present invention. In one embodiment, each puff releases or administer from about 1 to about 8 mg, or from about 2 to about 6 mg or from about 3 to about 5 mg or about 4 mg of the composition of the present invention. Thus according to some embodiments, the dose comprises 2, 3, or 4 puffs, wherein each puff releases from about 2 to about 6 mg of the composition.

According to one embodiment, the composition or the pharmaceutical composition of the present invention is administer to a human subject. According to another embodiment, the subject is a non-human mammal such as livestock animals, domestic pets, rodents, lagomorpha and primates. In one embodiment the livestock animals is selected from cattle, pigs, sheep, goats, horses, mules, donkey, buffalo, and camels. In some other embodiments, the domestic pet is a cat or dog, the rodent is rat, mice guinea pig or hamster, the lagomorpha is a rabbit, and the primate is monkey such as macaques or ape such as chimpanzee.

The term “sleep disorder” refers to a disruptive pattern of sleep arising from many causes. Examples of sleep disorders which may be treated using the composition of the present invention are insomnia (e.g., transient, short-term, and chronic), delayed sleep phase syndrome, hypnotic-dependent sleep disorder, and stimulant-dependent sleep disorder; disorders associated with difficulties in adhering to a regular sleep schedule such as sleep state misperception, shift work sleep disorder, chronic time zone change syndrome, and irregular sleep-wake syndrome; disorders associated with abnormal behaviors such as sleep terror disorder (i.e., parasomnia) and sleepwalking (i.e., somnambulism); and other disorders such as sleep bruxism, fibromyalgia, and nightmares; dyssomnia circadian rhythm sleep disorders, and parasomnia. In some embodiments, sleep disorder is dyssomnia such as insomnia.

According to some embodiments, the composition of the present invention comprising CBN and at least one sleep inducing terpenes has a synergic effect on inducing the sleeping, i.e. the sleeping effect of the composition of the present invention comprising CBN and at least one terpene is synergistic. As use herein, the term “synergistic effect” means that the combination of the components of a composition exhibits greater effect or activity than the additive effect or activity provided when each component of the combination is applied alone. The synergistic effect was amply demonstrated in the examples using rodent model and can be easily applied for any subject using conventional and well known conversion factors.

The extract of cannabis of the present invention may be obtained by any known method of extraction. Non-limiting extraction method, suitable according to the present invention, is described in the Examples. According to one embodiment, the method comprises extraction form a cannabis plant and addition of additional cannabinoids and/or additional terpenes to said extract to obtain the composition of the present invention. Thus, in one embodiment, the present invention provides a composition prepared by extraction of cannabis plant and addition of (i) one or more additional cannabinoid, (ii) one or more additional terpenes, or (iii) combination of (i) and (ii). The extraction may effected using any known method as described in the examples. Commercial extracts may be used as well. The composition may be prepared by direct filling the extract into a container or by mixing the desired cannabinoids fraction(s) with the desired terpenes in closed container protected from light and then used for filling the vaporizer cartridges. The composition of the present invention may be prepared by mixing the purified cannabinoids and terpenes in the desired proportions.

According to another aspect, the present invention provides a container comprising the composition of the present invention. According to some embodiments, the container is suitable for use with a vaporizer, for example with using cartomizers, atomizers or clearomizer type vaporizer. According to some embodiments, the container, cartridge or tank containing the composition is a disposable or refillable container. According to other embodiments, the container is suitable or configured to use with a vaporizer utilizing a wick and coil heating element or utilizing a heating chamber such as ceramic heating chamber or titanium/quartz heating chamber. According to some embodiments, the container is a wick and coil container or a ceramic container. According to some embodiments, the ceramic container is a VapePod container using the technology called CCELL Ceramic Technology. In an ordinary atomizer to coil resides on the surface of the wick causing inconsistent heating. Additionally, ordinary wick designs cause reduced flow to the heating coil. The heating coil is embedded within the ceramic core ensures the atomizer is uniformly heated. According to some embodiments, the container is a wick and coil container or a ceramic container.

According to yet another aspect, the present invention provides a method for treating a sleep disorder in a subject in need thereof, said method comprising administering via vaporization the composition of the present invention. Administration via vaporization contemplates vaporization of the composition using a vaporizer and then inhaling the vapors. According to one embodiment, the present invention provides a method for inducing sleep in a subject in need thereof, said method comprising administering via vaporization the composition of the present invention. According to yet another embodiment, the present invention provides a method for increasing sleep duration in a subject in need thereof, said method comprising administering via vaporization the composition of the present invention. According to one embodiment, the subject is human. According to another embodiment, the subject is a non-human mammal such as domestic pet, e.g. cat or dog.

The term “treating” a condition or patient refers to taking steps to obtain beneficial or desired results, including clinical results. Beneficial or desired clinical results include, but are not limited to, alleviation or amelioration of one or more symptoms of a sleep disorder or parameters associated with it.

According to some embodiment, the sleep disorder is selected from insomnia (e.g., transient, short-term, and chronic), delayed sleep phase syndrome, hypnotic-dependent sleep disorder, and stimulant-dependent sleep disorder; disorders associated with difficulties in adhering to a regular sleep schedule such as sleep state misperception, shift work sleep disorder, chronic time zone change syndrome, and irregular sleep-wake syndrome; disorders associated with abnormal behaviors such as sleep terror disorder (i.e., parasomnia) and sleepwalking (i.e., somnambulism); and other disorders such as sleep bruxism, fibromyalgia, and nightmares; dyssomnia circadian rhythm sleep disorders, and parasomnia. In some embodiments, sleep disorder is dyssomnia such as insomnia.

The method of treating according to the present invention comprises administering the composition of the present invention via vaporization. According to one embodiment, the vaporization is performed using a vaporizer device. Any suitable vaporizer device is contemplated. In one particular embodiment, the vaporizer is a cartomizer, atomizer or clearomizer type vaporizer. According to some embodiments, the administration is made using a container adapted for use with a vaporizer, said container comprising the composition of the present invention.

According to some embodiments, the method comprises administering of about 1 to about 40 mg/day of CBN. According to other embodiments, treating comprises administering about 10 mg/ day to 140 mg/day, about 20 mg/day to about 120 mg/day, about 30 mg/day to about 100 mg/day, about 40 mg/day to about 80 mg/day or about 50 mg/day to about 60 mg/day. According to one embodiment, treating comprises administering of from 1 to 40 mg/day, from 40 to 80 mg/day or from 80 to 120 mg/day. According to some embodiments, the method comprises administering about 1.5 to 30 mg/day, about 2 to 25 mg/day, about 3 to about 20 mg/day, about 4 to about 15 mg/day or about 5 to about 10 mg/day of CBN. According to other embodiments, the method comprises administering about 1 to about 10 mg/day, about 2 to about 8 mg/day, or about 4 to about 6 mg/day of CBN. According to some embodiments, the method comprises administering 1, 2, 3 or 5 mg of CBN 1, 2, 3 or 4 times a day. According to other embodiments, the method comprises administering 10 mg of CBN 1, 2, 3 or 4 times a day. According to other embodiments, treating comprises administering 40 mg one a day, or administering 15, 20 or 30 mg 2 times a day or administering 40 mg 3 times a day. According to one embodiment, the method comprises administering of about 1 to about 10 mg of the composition of the present invention 1, 2, 3 or 4 times a day. According to another embodiment, treating comprises administering of about 2 to about 8 mg of the composition of the present invention 1, 2, 3 or 4 times a day. According to still another embodiment, treating comprises administering of about 3 to about 6 mg of the composition of the present invention 1, 2, 3 or 4 times a day.

According to some embodiments, the composition of the present invention comprising CBN and at least one sleep inducing terpenes has a synergic effect on inducing the sleeping, i.e. the sleeping effect of the mixture of CBN with at least one terpene is synergistic. Administering can be performed, for example, once, a plurality of times for examples several puffs per administration, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to some embodiments, the composition is administered via a vaporizer or e-cigarette having a mechanism to control the volume of the vapor.

According to another aspect, the present invention provides a vaporizable composition comprising solely compounds naturally present in cannabis plants wherein the composition comprises from about 60% to about 98% of cannabinoids, and from about 2% to about 20% terpenes, and wherein the composition has the viscosity in the range of about 10 cP to about 2000 cP at 60° C. According to one embodiment, the composition comprises from about 80% to about 98% of cannabinoids, and from about 2% to about 20% terpenes. According to some embodiments, such vaporizable compositions of the present invention comprises high content of terpenes. According to one embodiment, all the terpenes originate from the cannabis extract or alternatively some or all terpenes are additionally added terpenes. In one embodiment, the terpenes are selected from limonene, α-pinene, β-myrcene, linalool, β-caryophyllene, carophyllene oxide, nerolidol, phytol, trans-β-ocimene, α-terpinolene, trans-caryophyllene, α-humulene, caryophyllene and any combination thereof. According to another embodiment, the additional one or more additional terpenes is selected from According to another embodiment, the terpene is selected from limonene, α-pinene, β-myrcene, linalool, β-caryophyllene, carophyllene oxide, cerolidol, phytol, linalyl acetate, borneol and any combination thereof According to one embodiment, the terpene is β-myrcene, and any combination thereof

According to another aspect, the present invention provides a composition comprising from about 1 wt % to about 30 wt % of cannabinol (CBN) and up to about 15 wt % of one or more terpenes, and wherein the composition has the viscosity in the range of about 10 cP to about 1000 cP at 60° C., wherein the composition is for preparation of a medicament for use in treating a sleep disorder.

According to some embodiments, the present invention provides a vaporizable composition comprising cannabis extract and optionally one or more additional terpenes naturally present in cannabis plant, wherein the composition comprises from about 80% to about 98% of cannabinoids, from about 2% to about 20% terpenes, and has the viscosity in the range of about 10 cP to about 2000 cP at 60° C. According to some embodiments, the composition comprises from about 85 wt % to about 98 wt % of cannabis extract. According to other embodiments, the composition comprises from about 10 wt % to about 85 wt % or from about 15 to about 60 wt % of cannabis extract. According to some embodiments, the composition has the viscosity in the range of about 100 cP to about 1000 cP at 60° C. According to one embodiment, the composition comprises cannabis extract and one or more additional terpene. According to another embodiment, the composition consists of cannabis extract and one or more additional terpene. According to one embodiment, the composition consists of the cannabis extract. According to another embodiment, the composition is substantially consists of the cannabis extract.

According to some embodiments, all the terpenes are originated from the extract. According to another embodiment, the terpenes of the composition are originated from the extract and from terpenes further added to the composition. According to an alternative embodiment, all the terpenes are originated from the additionally added terpenes. According to any one of the embodiments, the additional terpenes are terpenes naturally occurring in cannabis plant. These terpenes are devoid of any compound or excipient that is not present in the cannabis plant.

According to one embodiment, the composition of the present invention consists of the cannabis extract and additional terpenes.

According to any one of the above embodiments, the composition has a viscosity enabling use of the composition with vaporizers. According to one embodiment, the composition has a viscosity in the range of 10 cP to about 2000 cP at 60° C. According to another embodiment, the composition has a viscosity in the range of 10 cP to about 1000 cP at 60° C. According to a further embodiment, the composition has a viscosity in the range of about 20 cP to about 950 cP, about 40 cP to about 900 cP, about 60 cP to about 850 cP, about 80 cP to about 800 cP, about 100 cP to about 750 cP, about 150 cP to about 700 cP, about 200 cP to about 650 cP, or about 300 to 700 at 60° C. According to another embodiment, the viscosity is in the range 200 cP to 800 cP, 300 cP to 700 cP or 400 cP to 600 cP at 60° C.

Without being bound by any particular theory, it is believed that the relative low viscosity is achieved by relatively high concentration of terpenes naturally occurring in cannabis plant and/or high purity and/or efficient removal or absence of waxes and lipids. According to some embodiments, the composition comprises terpenes in the range of about 2 wt % to about 30 wt %. According to one embodiment, the composition comprises about 3 to about 25 wt %, about 4 to about 20 wt %, about 5 to about 18 wt %, about 6 to about 15 wt %, about 7 to about 12 wt %, or about 8 to about 10 wt % of terpenes. According to some embodiment, the composition comprises about 5 to about 20 wt %, about 7 to about 18 wt %, about 10 to about 15 wt % of terpenes. According to other embodiment, the composition comprises at least 2 wt %, at least 5 wt %, at least 7 wt %, at least 10 wt %, at least 12 wt %, at least 15 wt % of terpenes. According to another embodiment, the composition comprises no more than 30 wt %, no more than 25 wt %, or no more than 20 wt % or no more than 10 wt %, or no more than 5 wt % of terpenes. The terms “no more” and “less than” may, in some embodiments, be used interchangeably. As disclosed already the terpenes may be terpenes originated from the cannabis extract, from additional terpenes added or from the combination of terpenes originated from the extract and of additional terpenes. According to any one of the above embodiments, the terpenes are terpenes naturally present in cannabis plants. According to one embodiment, the terpenes are selected from limonene, α-pinene, β-myrcene, linalool, β-caryophyllene, carophyllene oxide, nerolidol, phytol, trans-β-ocimene, α-terpinolene, trans-caryophyllene, α-humulene, caryophyllene and combinations thereof. According to other embodiments, the terpenes may include any other terpenes identified in the cannabis variety herbs.

According to any one of the above embodiment, the composition comprises about 80 wt % to about 98 wt % cannabinoids. According to some embodiments, the composition comprises from 82 wt % to about 96 wt %, about 84 wt % to about 94 wt %, about 86 wt % to about 92 wt %, or about 88 wt % to about 90 wt % of cannabinoids. According to one embodiment, the cannabinoids comprise one or more of the cannabinoids delta-8-tetrahydrocannabinol, delta-9-tetrahydrocannabinol (THC), cannabidiol (CBD), cannabinol (CBN), cannabigerol (CBG), delta-9-tetrahydro cannabinotic acid, the nonpsychotropic cannabinoid 3-dimethylnepty 11 carboxylic acid homologine 8, delta-8-tetrahydrocannabinol.

Other non- limiting examples of cannabinoids are Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM). According to some embodiments, the composition comprises CBN. According to one embodiment, the composition comprises from about 1 wt % to about 18 wt % of cannabinol. In other embodiments, the composition comprises from about 2 wt % to about 16 wt %, about 3 wt % to about 14 wt %, about 4 wt % to about 12 wt %, or about 6 wt % to about 10 wt % of CBN. In further embodiments, the composition comprises from about 2 wt % to about 10 wt %, or from about 4 wt % to about 8 wt %, or from about 5 to about 6 wt % of CBN. In certain embodiments, the composition comprises from about 3 wt % to about 15 wt %, or about 5 wt % to about 10 wt % of CBN. According to yet another embodiment, the composition comprises less than 15 wt %, or less than 10 wt %, or less than 8 wt % of CBN.

According to some embodiment, the weight ratio of terpenes to cannabinoids is in the range of about 1:50 to 1:4 or about 1:49 to about 1:10. According to another embodiment, the weight ratio of terpenes to cannabinoids is in the range of 1:30 to 1:5, 1:20 to 1:7 or 1:15 to 1:10. According to some embodiments, the weight ratio of terpenes to cannabinoids is in the range 1:8 to 1:12.

According to some embodiments, the composition comprises about 80 to 95 wt % cannabinoids and about 5 to about 20 wt % of terpenes. According to one embodiment, the composition comprises about 85 to 95 wt % of cannabinoids and about 5 to about 15 wt % of terpenes. According to some embodiments, such composition consists of cannabis extract and additional terpenes. According to some embodiment, the additional terpenes are selected from limonene, α-pinene, β-myrcene, linalool, β-caryophyllene, carophyllene oxide, nerolidol, phytol, and combinations thereof

According to any one of the above embodiments, the composition comprises cannabis extract, e.g. 80 to 99 or to 98 wt % of cannabis extract. According to one embodiment, the composition comprises about 82 to 98 wt %, about 85 to about 95 wt % or from about 87 to about 92 wt % of cannabis extract. According to another embodiment, the composition comprises about 90 wt % to about 98 wt %, about 92 wt % to about 97 wt % or about 94 wt % to about 96 wt % of extract. According to some embodiments, the cannabis extract comprises from about 80 wt % to about 98 wt % of cannabinoids and from about 2 wt % to about 20 wt % of terpenes. According to one embodiment, the extract comprises from about 82 to 98 wt %, from about 85 to about 95 wt % or from about 87 to about 92 wt % of cannabinoids. According to some embodiments, the extract comprises about 90 wt % to about 99 wt %, about 92 wt % to about 98 wt % or about 94 wt % to about 96 wt % of cannabinoids. According to another embodiment, the extract comprises at least 85 wt %, at least 90 wt %, at least 92 wt % or at least 94 wt % of cannabinoids. According to another embodiment, the extract comprises from about 2 to 20 wt %, about 4 to about 18 wt %, about 8 to about 16 wt %, about 6 to about 14 wt %, about 8 to about 12 wt % of terpenes. According to further embodiment, the extract comprises about 1 wt % to about 10 wt %, about 2 wt % to about 9 wt %, about 3 wt % to about 8 wt %, about 4 wt % to about 7 wt %, or about 5 wt % to about 6 wt % of terpenes.

According to any one of the above embodiments, the composition has a peak of absorbance at 293 nm. According to some embodiments, the absorbance is measured upon dilution of the composition in ethanol. According to some embodiments, the absorbance of the composition at 293 nm is less than 3 absorbance units (AU), less than 2 AU, less than 1 AU or less than about 0.5 AU, when measured upon 1 to 100 dilution of the composition in ethanol.

It is contemplated that according to some embodiments, the composition of the present invention consists of cannabis extract. According to other embodiments, the composition consists of cannabis extract and may comprise additional terpenes naturally present in the cannabis plant. However, the composition of the present invention is devoid of any compound, excipient or additive, which is not present in the cannabis plant. It is known in the art to add additives to cannabis extract to accomplish certain properties, such as viscosity, in order to be able to use these enriched extracts with electronic cigarettes. Such additives are usually are not healthy and should be avoid. The term “additive” as used herein refers to any compound added to a composition, which is not naturally present in cannabis plants. The main advantage of the composition of the present invention is that the composition consists of only compound naturally present in the cannabis plants and devoid of any compound that is not present or absent from the plants. It should be understood that the term devoid may comprise composition in which traces of compounds used in the process of purification are present. Nevertheless, the amount of such compounds is neglectable, e.g. the amount of such compound is less than 0.001 wt % or less than 0.0001 wt % of the composition. Thus, according to any one of the above embodiments, the composition is devoid of any additives. According to some embodiments, the additive is selected from the group consisting of oils, lipids, waxes, waxes esters, propylene glycol, butylene glycol, hexylene glycol, glycerin, polyethylene glycol, organic solvent, vitamin E and any combinations thereof. According to some embodiment, the oils are selected from natural oils and synthetic oils. Such oils exclude oils naturally present in cannabis plants. According to another embodiment, glycerin is a vegetable glycerin. According for yet another embodiment, organic solvent is selected from ethanol, propanol, butanol, and isopropanol. In one embodiment, the composition is devoid of glycerin and in particular devoid of vegetable glycerin. In another embodiment, the composition is devoid of propylene glycol, butylene glycol, and hexylene glycol and the like.

According to one embodiment, the composition comprises cannabis extract comprising about 6 wt % to about 15 wt % terpenes, about 85 wt % to about 94 wt % cannabinoids, wherein the viscosity of the composition is in the range of about 10 cP to about 1000 cP at 60° C. or about 100 cP to 700 cP at 60° C., and said composition is devoid of additives.

According to another embodiment, the composition comprises cannabis extract and additional terpenes naturally present in cannabis plant, wherein the composition comprises about 6 wt % to about 15 wt % terpenes, about 85 wt % to about 94 wt % cannabinoids, wherein the viscosity of the composition is in the range of about 10 cP to about 1000 cP at 60° C., or about 100 cP to 700 cP at 60° C., and said composition is devoid of additives. According to another embodiment, the composition comprises about 8 wt % to about 10 wt % terpenes and about 90 wt % to about 92 wt % cannabinoids. According to some embodiments, the composition has an absorbance of less than 2 AU or less than 1 AU at 293 nm, when measured upon 1:100 dilution in ethanol.

In any one of the above embodiments, the term comprising has the meaning of consisting, thus in some embodiments, the present invention provides a vaporizable composition consisting of compounds naturally present in cannabis plants, wherein the composition comprises from about 80% to about 98% of cannabinoids and from about 2% to about 20% terpenes, and has the viscosity in the range of about 10 cP to about 1000 cP at 60° C.

According to other embodiments, the composition consists of cannabis extract and optionally one or more additional terpenes naturally present in cannabis plant, wherein the composition comprises from about 80% to about 98% of cannabinoids, from about 2% to about 20% terpenes, and has the viscosity in the range of about 10 cP to about 1000 cP at 60° C. According to one embodiment, the composition consists of cannabis extract comprising about 6 wt % to about 15 wt % terpenes, about 85 wt % to about 94 wt % cannabinoids, wherein the viscosity of the composition is in the range of about 100 cP to about 1000 cP at 60° C., 100 cP to 700 cP or 300 cP to 800 cP at 60° C., and said composition is devoid of additives. According to another embodiment, the composition consists of cannabis extract and additional terpenes naturally present in cannabis plant, wherein the composition comprises about 6 wt % to about 15 wt % terpenes, about 85 wt % to about 94 wt % cannabinoids, wherein the viscosity of the composition is in the range of about 200 cP to about 800 cP at 60° C. or 100 cP to 700 cP at 60° C., and said composition is devoid of additives. According to another embodiment, the composition comprises about 8 wt % to about 10 wt % terpenes and about 90 wt % to about 92 wt % cannabinoids. According to some embodiments, the composition has absorbance less of than 2 AU or less than 1 AU at 293 nm when measured upon 1:100 dilution in ethanol.

According to any one of the above embodiment, the composition is suitable for use with a container or cartridge configured to be used with a vaporizer. In some embodiments, the vaporizer are cartomizers, atomizers and clearomizer. According to some embodiments, the vaporizer utilizes a wick and coil heating element or a heating chamber such as ceramic heating chamber or titanium/quartz heating chamber. According to some embodiment, the container, cartridge or tank containing the composition is a disposable or refillable container, cartridge or tank. According to any one of the above embodiments, the composition is suitable for using with a container or cartridge configuration and vaporizer or e-cigarette that has a mechanism of control of volume of the vapor.

According to any one of the above embodiments, the composition is a pharmaceutical composition.

According to some embodiment, the composition or the pharmaceutical composition is for use in treating a disease or disordered treatable with cannabinoids. According to some embodiments, the disease or disorder may be, for example, neurological conditions and psychiatric disorders. According to some embodiments, the neurological conditions and psychiatric disorders are selected from anxiety disorders, PTSD, addiction, alcoholism, OCD, amyotrophic lateral sclerosis (ALS), arachnoid cysts, attention deficit/hyperactivity disorder (ADHD), autism, bell's palsy, bipolar disorder, brain/spinal cord tumors, carpal tunnel syndrome, catalepsy, cervical spondylosis, depression, dizziness, encephalitis, epidural abscess, epilepsy/seizures, extradural hemorrhage, sleep disorders, Guillain-Barre syndrome, headache, hematoma, infection, locked-in-syndrome, meningitis, migraine, multiple sclerosis, myelopathy, neuralgia, neurodegenerative disorders such as Alzheimer's disease, Huntington's disease and Parkinson's disease; peripheral neuropathy, polio, spinal cord injury, stroke, subarachnoid hemorrhage, subdural hemorrhage, Tourette's, transient ischemic attack (TIA), and schizophrenia/mental disorders. According to some embodiments, the disease or disorder is selected from anxiety, PTSD, pain, epilepsy, depression, migraines, cluster headache, bipolar disorder, multiple sclerosis, Parkinson's disease, Alzheimer's disease, Huntington disease, and Tourette syndrome. According to other embodiments, the disease or disorder is selected from anorexia, arthritis, asthma, atherosclerosis, atrophie blanche, back pain, cancer such as breast, colorectal, glioma/brain, leukemia, skin, lung, melanoma, oral, pancreatic, prostate, skin and testicular cancer, cerebral palsy, chronic cystitis, chronic pain, chronic stress, colon cancer, COPD, Crohn's disease, dermatitis, diabetes, disc degeneration, dystonia, endocannabinoid deficiency, fibromyalgia, glaucoma, heart disease, hepatitis, herpes, hiccups, HIV/AIDS, idiopathic intracranial hypertension, liver disease, lupus, malaria, Meige's syndrome, migraines, muscular dystrophy, nausea & vomiting, neuropathic pain, obesity, osteoporosis, painful bladder syndrome, pancreatic cancer, pruritis, sickle cell disease, spasticity, spinal cord injury, stroke, substance abuse, and Wilson's disease.

The extract of cannabis of the present invention may be obtained by any known method of extraction. Non-limiting extraction method, suitable according to the present invention, is described in the Examples. According to one embodiment, the method comprises extraction form a cannabis plant and addition of additional cannabinoids and/or additional terpenes to said extract. Thus, in one embodiment, the present invention provides a composition prepared by extraction of cannabis plant and addition of (i) one or more additional cannabinoid, (ii) one or more additional terpenes, or (iii) combination of (i) and (ii). The extraction may effected using any known method as described in the examples. The composition may be prepared by direct filling the extract into a container or by mixing the desired cannabinoids fraction(s) with the desired terpenes in closed container protected from light and then used for filling the vaporizer cartridges. The composition of the present invention may be prepared by mixing the purified cannabinoids and terpenes in the desired proportions.

According to another embodiment, the present invention provides a container comprising a vaporizable composition comprising solely compounds naturally present in cannabis plants wherein the composition comprises from about 60% to about 98% of cannabinoids, and from about 2% to about 20% terpenes, and wherein the composition has the viscosity in the range of about 10 cP to about 2000 cP at 60° C. According to some embodiments, the container is suitable for use with a vaporizer, for example with using cartomizers, atomizers or clearomizer type vaporizer. According to some embodiments, the container, cartridge or tank containing the composition is a disposable or refillable container. According to other embodiments, the container is suitable or configured to use with a the vaporizer utilizing a wick and coil heating element or utilizing a heating chamber such as ceramic heating chamber or titanium/quartz heating chamber. According to some embodiments, the ceramic container is a VapePod container using the technology called CCELL Ceramic Technology. In an ordinary atomizer to coil resides on the surface of the wick causing inconsistent heating. Additionally, ordinary wick designs cause reduced flow to the heating coil. The heating coil is embedded within the ceramic core ensures the atomizer is uniformly heated. According to some embodiments, the container is a wick and coil container or a ceramic container.

According to another embodiment, the present invention provides a method for treating a disease or disordered treatable with cannabinoids in a subject in need thereof, said method comprising administering via vaporization the composition comprising solely compounds naturally present in cannabis plants wherein the composition comprises from about 60% to about 98% of cannabinoids, and from about 2% to about 20% terpenes, and wherein the composition has the viscosity in the range of about 10 cP to about 2000 cP at 60° C. Administration via vaporization contemplates vaporization of the composition using a vaporizer and then inhaling the vapor.

In some embodiments, the method comprising administering a vaporizable composition comprising cannabis extract and optionally one or more terpenes naturally present in cannabis plant, wherein the composition comprises from about 80% to about 98% of cannabinoids, from about 2% to about 20% terpenes, and has the viscosity in the range of about 10 cP to about 1000 cP at 60° C. According to some embodiments, the disease or disorder may be, for example, neurological conditions and psychiatric disorders. According to some embodiments, the neurological conditions and psychiatric disorders are selected from anxiety disorders, PTSD, addiction, alcoholism, OCD, amyotrophic lateral sclerosis (ALS), arachnoid cysts, attention deficit/hyperactivity disorder (ADHD), autism, bell's palsy, bipolar disorder, brain/spinal cord tumors, carpal tunnel syndrome, catalepsy, cervical spondylosis, depression, dizziness, encephalitis, epidural abscess, epilepsy/seizures, extradural hemorrhage, sleep disorders, Guillain-Barre syndrome, headache, hematoma, infection, locked-in-syndrome, meningitis, migraine, multiple sclerosis, myelopathy, neuralgia, neurodegenerative disorders such as Alzheimer's disease, Huntington's disease and Parkinson's disease; peripheral neuropathy, polio, spinal cord injury, stroke, subarachnoid hemorrhage, subdural hemorrhage, Tourette's, transient ischemic attack (TIA), and schizophrenia/mental disorders. According to some embodiments, the disease or disorder is selected from anxiety, PTSD, pain, epilepsy, depression, migraines, cluster headache, bipolar disorder, multiple sclerosis, Parkinson's disease, Alzheimer's disease, Huntington disease, and Tourette syndrome. According to other embodiments, the disease or disorder is selected from anorexia, arthritis, asthma, atherosclerosis, atrophie blanche, back pain, cancer such as breast, colorectal, glioma/brain, leukemia, skin, lung, melanoma, oral, pancreatic, prostate, skin and testicular cancer, cerebral palsy, chronic cystitis, chronic pain, chronic stress, colon cancer, COPD, Crohn's disease, dermatitis, diabetes, disc degeneration, dystonia, endocannabinoid deficiency, fibromyalgia, glaucoma, heart disease, hepatitis, herpes, hiccups, HIV/AIDS, idiopathic intracranial hypertension, liver disease, lupus, malaria, Meige's syndrome, migraines, muscular dystrophy, nausea & vomiting, neuropathic pain, obesity, osteoporosis, painful bladder syndrome, pancreatic cancer, pruritis, sickle cell disease, spasticity, spinal cord injury, stroke, substance abuse, and Wilson's disease.

The composition of the present invention is administered via vaporizing device. Any suitable device may be used. According to one embodiment, the vaporizing device is of cartomizers, atomizers or clearomizer type, preferably clearomizer. According to some embodiments, the composition is administered via a vaporizer or e-cigarette having a mechanism to control the volume of the vapor. In some embodiments, the method comprises delivering in each inhalation about 2 mg of cannabinoids, whereas repeated inhalation deviates no more than 50%, or no more than 40%, or no more than 30% of the median inhaled amount of cannabinoids vaporized in one action of inhalation. In other embodiments, repeated inhalation deviates no more than 20% or no more than 10% of the median inhaled amount of cannabinoids vaporized in one action of inhalation.

The term “comprising” always includes the term “consisting” and when the term “comprising” appears in an embodiment of the disclosure, this same embodiment wherein the term “consisting” replaces the term “comprising” is always also an embodiment of the disclosure.

As used herein, the term “about”, when referring to a measurable value such as an amount, a temporal duration, and the like, is meant to encompass variations of +/−10%, or +/−5%, +/−1%, or even +/−0.1% from the specified value.

According to some embodiments, the term “about 0 wt %” refer to 0.0001 wt %, 0.001 wt %, 0.01 wt % or 0.1 wt % or that the compound is absent.

Having now generally described the invention, the same will be more readily understood through reference to the following examples, which are provided by way of illustration and are not intended to be limiting of the present invention.

### EXAMPLES

### Example 1. Production of 150 gr of the Composition

1 kg of flowers of cannabis was place into a closed loop extraction system used for solvent decomposing fluid butane. In certain process, the butane fluid flow through the closed loop system where the cannabis material is sealed. When the all plant material was soaked, a valve shuted the flow to soak for a period of 5 to 20 minutes. After this soaking period the valve was open for the solvent fluid to flow in to the collecting tank. Alternative extraction fluids or solvents were hexane, pentane, cyclopentane n-butane, ISO butane, CO2 and nitrogen and combinations thereof

Once all fluids extracted in the collecting tank, recovery at room temperature occurred, the butane separated from the oil to go back to the solvent tank and the oil stayed in the collecting tank. Once the butane was recovered, the oil was collected from the extraction device and winterized in alcohol for 48 hours at the temperature of between −15 to −25° C. and then filtered to separate waxes from oleoresin. Once winterized, the batch was mixed with alcohol and processed through a rotavapor machine to evaporate the alcohol. Once the alcohol evaporated, the oil was places in a short path distillation system, and the fractionation of molecule started.

We started with terpenoid at an appropriate temperature for amount of time necessary until the flow stopped.

We then stepwise increased the temperature for extracting each cannabinoid at its respective boiling point, and collected in different flask each extraction until the flow stopped.

The process was repeated multiple times to refine the batch. Different extraction batches are presented in Table 5.

TABLE 5
Different extraction batches

| | Comp A | Comp B | Comp C | Comp D | Comp E | Comp F | Comp G |
|---|---|---|---|---|---|---|---|
| Ingredients | % W/W | % W/W | % W/W | % W/W | % W/W | % W/W | % W/W |
| CBN | 5 | 5 | 10 | 10 | 15 | 15 | 15 |
| CBD | 70 | 70 | 45 | 45 | 20 | 20 | 20 |
| THC | 10 | 10 | 32 | 32 | 54 | 54 | 55 |
| Other cannabinoids | 6 | 3 | 6 | 3 | 6 | 3 | 6 |
| CBG, CBC, CBV, THCA, CBDA | | | | | | | |
| Myrcene | 5 | 5 | 3 | 3 | 1 | 1 | 1 |
| Other terpenes | 3 | 6 | 3.5 | 6.5 | 3.8 | 6.8 | 3.8 |
| Residual solvents and ash matter | 1 | 1 | 0.5 | 0.5 | 0.2 | 0.2 | 0.2 |
| Total | 100 | 100 | 100 | 100 | 100 | 100 | 100 |

In one arrangement, the purified cannabinoids obtained from the extraction were mixed at a predefined proportions to provide the composition as described in Tables 6 and 7.

TABLE 6
Compositions comprising different combinations of cannabinoids and terpenes

| Formulation | A1 | A2 | A3 | A4 | A5 | A6 | A7 | A8 | A9 | A10 | A11 |
|---|---|---|---|---|---|---|---|---|---|---|---|
| Ingredients | wt % | wt % | wt % | wt % | wt % | wt % | wt % | wt % | wt % | wt % | wt % |
| Pure CBD 98% | 73 | 65 | 67 | 80 | 73 | 65 | 67 | 80 | 65 | 72 | 72 |
| Pure CBN 98% | 7 | 14 | 20 | 0 | 7 | 15 | 20 | 0 | 19 | 12 | 11 |
| Pure THC 90% | 10 | 11 | 0 | 0 | 10 | 10 | 0 | 0 | 10 | 0 | 0 |
| Pure CBG 98% | 0 | 0 | 3 | 10 | 0 | 0 | 3 | 10 | 0 | 4 | 5 |
| Pure CBC 99% | 0 | 0 | 0 | 0 | 0 | 0 | 0 | 0 | 0 | 2 | 2 |
| Linalool | 0 | 0 | 0 | 0 | 10 | 10 | 10 | 10 | 3 | 5 | 5 |
| Myrcene | 10 | 10 | 10 | 10 | 0 | 0 | 0 | 0 | 3 | 5 | 5 |
| Total | 100 | 100 | 100 | 100 | 100 | 100 | 100 | 100 | 100 | 100 | 100 |

TABLE 7
Compositions comprising different combinations of cannabinoids and terpenes

| Formulation | A12 | A13 | A14 | A15 | A16 | A17 | A18 | A19 | A20 | A21 |
|---|---|---|---|---|---|---|---|---|---|---|
| Ingredients | wt % | wt % | wt % | wt % | wt % | wt % | wt % | wt % | wt % | wt % |
| Pure CBD 98% | 79 | 62 | 66 | 65 | 67 | 79 | 62 | 66 | 65 | 67 |
| Pure CBN 98% | 5 | 20 | 15 | 12 | 5 | 10 | 20 | 15 | 12 | 5 |
| Pure THC 90% | 0 | 6 | 7 | 10 | 15 | 0 | 6 | 7 | 10 | 15 |
| Pure CBG 98% | 6 | 2 | 0 | 0 | 3 | 6 | 2 | 0 | 0 | 3 |
| Pure CBC 99% | 0 | 0 | 2 | 3 | 0 | 0 | 0 | 2 | 5 | 0 |
| Linalool | 3 | 7 | 5 | 2 | 2 | 1 | 0 | 4 | 0 | 0 |
| Myrcene | 3 | 1 | 0 | 4 | 2 | 0 | 0 | 0 | 0 | 0 |
| Borneol | 2 | 1 | 0 | 1 | 2 | 2 | 0 | 1 | 4 | 0 |
| Nerolidol | 2 | 0 | 3 | 2 | 2 | 2 | 7 | 2 | 4 | 10 |
| Terpinolene | 0 | 1 | 2 | 1 | 2 | 0 | 3 | 3 | 0 | 0 |
| Total | 100 | 100 | 100 | 100 | 100 | 100 | 100 | 100 | 100 | 100 |

In an additional arrangement, we mixed a cannabis extract from particular stains with cannabinoids and terpenes obtained from cannabis extract. The compositions are presented in Tables 8 and 9

TABLE 8
Compositions comprising extracts and combinations of cannabinoids and terpenes

| Formulation | B1 | B2 | B3 | B4 | B5 | B6 | B7 | B8 | B9 | B10 | B11 |
|---|---|---|---|---|---|---|---|---|---|---|---|
| Ingredients | wt % | wt % | wt % | wt % | wt % | wt % | wt % | wt % | wt % | wt % | wt % |
| Avidekel ™ BHO | 42 | 0 | 0 | 0 | 0 | 0 | 0 | 0 | 10 | 20 | 0 |
| extract | | | | | | | | | | | |
| Tachllta Till ™ BHO | 0 | 30 | 0 | 0 | 15 | 15 | 15 | 5 | 5 | 5 | 10 |
| extract | | | | | | | | | | | |
| Blue Sky ™ BHO | 0 | 0 | 33 | 0 | 15 | 15 | 15 | 10 | 10 | 10 | 10 |
| extract | | | | | | | | | | | |
| Jerusalem Espress ™ | 0 | 0 | 0 | 50 | 15 | 15 | 15 | 20 | 20 | 20 | 5 |
| BHO extract | | | | | | | | | | | |
| Pure CBD 99% | 32 | 49 | 49 | 38 | 38 | 44 | 43 | 51 | 46 | 26 | 58 |
| Pure CBN 99% | 20 | 14 | 8 | 4 | 11 | 5 | 7 | 9 | 5 | 15 | 11 |
| Linalool | 3 | 2 | 5 | 8 | 1 | 3 | 1 | 4 | 2 | 2 | 1 |
| Myrcene | 3 | 5 | 5 | 0 | 5 | 3 | 4 | 1 | 2 | 2 | 5 |
| Total | 100 | 100 | 100 | 100 | 100 | 100 | 100 | 100 | 100 | 100 | 100 |

TABLE 9
Compositions comprising extracts and combinations of cannabinoids and terpenes

| Formulation | B12 | B13 | B14 | B15 | B16 | B17 | B18 | B19 | B20 | B21 |
|---|---|---|---|---|---|---|---|---|---|---|
| Ingredients | wt % | wt % | wt % | wt % | wt % | wt % | wt % | wt % | wt % | wt % |
| Avidekel ™ BHO | 0 | 0 | 0 | 10 | 20 | 0 | 0 | 0 | 10 | 20 |
| extract | | | | | | | | | | |
| Tachllta Till ™ BHO | 10 | 10 | 5 | 5 | 5 | 10 | 10 | 5 | 5 | 5 |
| extract | | | | | | | | | | |
| Blue Sky ™ BHO | 9 | 8 | 0 | 10 | 10 | 14 | 8 | 7 | 15 | 20 |
| extract | | | | | | | | | | |
| Jerusalem Espress ™ | 5 | 0 | 10 | 16 | 20 | 5 | 5 | 10 | 16 | 20 |
| BHO extract | | | | | | | | | | |
| Pure CBD 99% | 65 | 70 | 70 | 50 | 30 | 65 | 70 | 70 | 50 | 30 |
| Pure CBN 99% | 5 | 7 | 10 | 5 | 11 | 0 | 2 | 3 | 0 | 1 |
| Linalool | 3 | 1 | 4 | 2 | 2 | 3 | 1 | 4 | 2 | 2 |
| Myrcene | 3 | 4 | 1 | 2 | 2 | 3 | 4 | 1 | 2 | 2 |
| Total | 100 | 100 | 100 | 100 | 100 | 100 | 100 | 100 | 100 | 100 |

Avidekel™ BHO extract comprises about 4 wt % THC, about 72 wt % CBD and about 2 wt % CBN, Tachllta Till™ BHO extract comprising about 76 wt % THC and about 2 wt % CBD, Blue Sky™ BHO extract comprising about 32 wt % of THC and about 0.4 wt % of CBD and Jerusalem Espress™ BHO extract comprising about 54 wt % THC about 2 wt % CBD and about 1 wt % CBN.

### Example 2. Administration of the Compositions to 5 Healthy Subjects

### Genetics

We have used 3 strains of cannabis plants obtained from the Isreali medical farm Teva Adir for preparation of the compositions.
Maui Waui (THC/CBD 10:1)
Royal Medic (THC/CBD 1:1)
Bob & Mary (THC/CBD 1:5)

### Formulation

For the initial human experiments, a composition as shown in Table 10 below was used.

TABLE 10
The composition used in the initial human experiment

| | Formulation |
|---|---|
| | Composition C1 |
| Ingredients | wt % |
| Pure CBD 98% | 65 |
| Pure CBN 98% | 14 |
| Pure THC 90% | 11 |
| Myrcene | 10 |

### Delivery System

We capsuled the formulations into our vaporizer cartridges (VapePod).

### Clinical Trials

We have provided one cartridge (500 mg) to each one of 5 healthy people. They were told to consume 4 inhalation before bed time; each inhalation was approximately 2 mg of the composition, the full dose before bed time was estimated as about 8 mg of the composition.

### Results

As can be seen from Table 11 presenting the result of the initial experiment, most of the subjects reported a fast falling asleep and good quality of sleep after administration of the composition.

TABLE 11
Effect of CBN comprising composition on the
speed of falling asleep and sleep quality.

| | Age and | Sleep | speed of |
|---|---|---|---|
| | Gender | quality | falling asleep |
| Patient #1 - | Male - 36 y | 4 | 3 |
| Patient #2 - | Male - 33 y | 3 | 3 |
| Patient #3 - | Female - 30 y | 3 | 3 |
| Patient #4 - | Female - 39 y | 4 | 4 |
| Patient #5 - | Male - 42 y | 3 | 2 |

1to 4—Sleep quality
0—very bad (didn't sleep much)
1—low quality
2—average sleep
3—above average
4—very good,
1to 4 —Speed of falling asleep
0—a few hours
1—an hour
2-30 min
3—very fast less than 30 min
4—immediately less than 10 min form lying in bed

In a further human experiments (safety experiment), a composition as shown in Table 12 below was prepared and used as described above. The results are shown in Table 13 (using the same scale as in Table 11).

TABLE 12
The composition used in second human experiment (safety)

| | Formulation |
|---|---|
| | Composition C2 |
| Ingredients | wt % |
| Tachllta Till ™ BHO extract | 15 |
| Blue Sky ™ BHO extract | 15 |
| Jerusalem Espress ™ BHO extract | 15 |
| Pure CBD 99% | 43 |
| Pure CBN 99% | 7 |
| Linalool | 1 |
| Myrcene | 4 |

TABLE 13
Results of the safety experiment

| | Age and | Sleep | speed of |
|---|---|---|---|
| | Gender | quality | falling asleep |
| Patient #1 - | Male - 37 y | 5 | 3 |
| Patient #2 - | Female - 31 y | 2 | 3 |
| Patient #3 - | Female - 41 y | 4 | 4 |
| Patient #4 - | Female - 69 y | 5 | 3 |
| Patient #5 - | Male - 75 y | 5 | 4 |

As can be seen from Table 13, most of the subjects reported a good quality of sleep and fast falling asleep. No sides effect were observed or reported.

### Example 3. Absorbance Measurement for the Extract Comp E

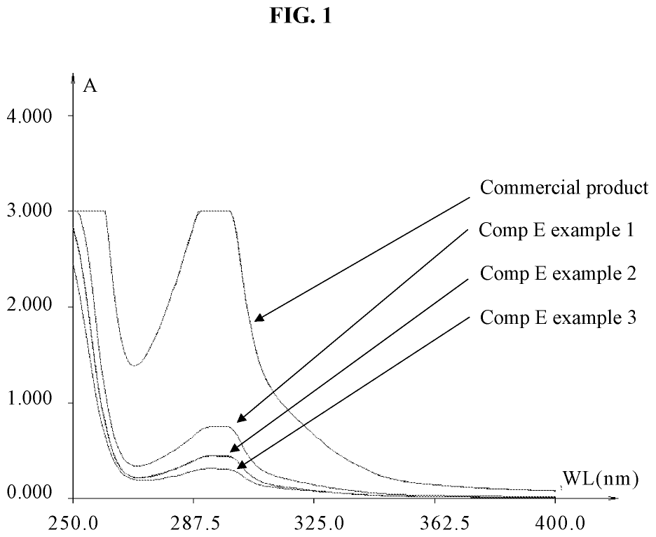

The obtained extract Comp E (Table 5) and an example of comercial product were diluted with ethanol and the UV-VIS absorbance between 250 nm to 400 nm was measured, when maximal absorbance was measured at 293 nm (see FIG. 1).

### Example 4. Administration of Measured Dose of the Extract

The amount in mg of a composition inhaled in each puff was measured. The dose was taken by inhalation of the extract using a dosing vaporizer. The vaporizer has a processor that indicates the start of the inhalation and immediately activates the heating element; after 2 seconds of inhalation (approx. 2mg) the device stopped heating, thus a metered dose of extraction was vaporized.

In our test samples, every puff provided a dose of about 2mg ±20% of the composition.

In an additional experiment using extraction vaporizer, each puff provided administration of about 4 mg ±20% of the composition.

### Example 5. Evaluation of a Combinations of Cannabinoids and Terpenes in Sleep Induction Model in Mice

The objective of the present study is to evaluate various cannabinoid mixtures with terpenes in the sleep induction model in mice and then in the second part optimize the doses.

Study Variables and End Points
Mortality and morbidity—once a day.
Body weight—during acclimation and before dosing.
Behavioral test—righting reflex—sleep latency and duration

The principle of the study is based on the ability of sleep promoting agents to shorten the time until Loss of Righting Reflex (LORR) occurs and prolong the presence of LORR in rodents treated with established anesthetizing agent.

Mice were be utilized and divided into 62 groups of three in each group. The number of groups and the total number of animals is based on previous studies demonstrating that this is the minimal number of animals sufficient to obtain indicative/significant information.

Combinations of 3 different cannabinoids: CBN, CBD and THC and 2 terpenes: (β-myrcene and linalool were tested. Diazepam (2 mg/kg) was used as a positive control. Ethanol:Cremophor:Saline (1:1:18) was used as negative control. The items were dissolved in Ethanol:Cremophor:Saline. The items, positive and negative controls were IP injected. Three (or four) mice were used for each group. 5 cycles of experiments were performed. In Cycles 1 each cannabinoid at different concentrations was separately administered; in Cycle 2—combinations of the cannabinoids with different concentration were administered (4 mice per group were used for items), in Cycle 3—different terpenes at different concentration were administered, in Cycle 4—combinations of different cannabinoids and terpenes were administered and in Cycle 5—compositions with high content of cannabinoids and low content of terpenes were tested (4 mice per group).

Female mice BALB/c strain of 7 weeks age were used for the experiment. The mice were allowed to acclimatize for at least 5 days. Animals were fed ad libitum a commercial rodent diet (Teklad Certified Global 18% Protein Diet, Harlan cat #2018). Animals had free access to sterilized and acidified drinking water (pH between 2.5 and 3.5) obtained from the municipality supply. Animals were housed under standard laboratory conditions, air conditioned and filtered (HEPA F6/6) with adequate fresh air supply (Minimum 15 air changes/hour). Animals were kept in a climate controlled environment. Temperatures range were 18-24° C. and relative humidity range 30-70% with a 12 hours light and 12 hours dark cycle (6 AM/6 PM).

### Experimental Procedure

The duration of the study was one day per cycle. The study was conducted in five cycles (see Tables 10-15). Each Test Item or vehicle formulation was administrated to three (or four) animals per group. Morbidity and mortality was checked once a day.

Sleeping Test was performed according to Pharmaseed's SOP No. 130: “Loss of Righting Reflex”. Mice were injected intraperitoneally (IP) with the positive control Diazepam (2 mg/kg, IP), vehicle or with the Test Items 30 minutes prior to Pentobarbital (40 mg/kg, IP) administration. Observation of the mouse begun immediately after administration of Pentobarbital. Once the mouse was asleep (the loss of righting reflex—LORR), it was be transferred to a home cage and placed on its back. Mice was considered awake when they could successfully upright themselves (all four feet in contact with the surface). Once a mouse righted itself, it was placed on its back once again and allowed to right a second time for confirmation. Expected sleep onset and duration of the negative control group was 5-8 min and 20-25 min, respectively. For positive control expected sleep onset and duration were 3-4 min and 40-50 min, respectively.

While the animals were asleep, fatty ophthalmic ointment (e.g. Duratears®) was be applied to their eyes to avoid drying of the eye. Also, since Diazepam might cause hypothermia, the cages with the sleeping mice were be placed on a heating pad.

The following parameters were be recorded and served as an index of sleep behavior:
Sleep latency—time required to sleep induction.
Sleep duration.

At termination, the animals were sacrificed by CO₂ asphyxiation.

The results are summarized in Tables 14-19.

TABLE 14
Cycle 1 - Effect of different cannabinoids
on sleep latency and duration

| | | | Sleep latency | | Sleep duration | |
|---|---|---|---|---|---|---|
| | | Dose | median | | median | |
| Group | Test item | mg/kg | (mean) | | (mean) | |
| 1F | Diazepam | 2 | 2 | (3) | 94 | (94) |
| 2F | Vehicle | — | No LORR | | No LORR | |
| 3F | CBN | 0.1 | 4 | (5) | 34 | (26) |
| 4F | | 0.5 | 8 | (8) | 15 | (14) |
| 5F | | 2 | 6 | (6) | 25 | (23) |
| 6F | | 5 | 8 | (9) | 32 | (31) |
| 7F | THC | 0.5 | 4 | (4) | 8 | (6) |
| 8F | | 2 | 20 | (20) | 0 | (12) |
| 9F | | 5 | 24 | (25) | 9 | (10) |
| 10F | | 10 | 11 | (11) | 11 | (21) |
| 11F | CBD | 1 | 16 | (13) | 9 | (14) |
| 12F | | 5 | 6 | (6) | 49 | (49) |
| 13F | | 20 | 9 | (8) | 48 | (48) |
| 14F | | 50 | 6 | (6) | 68 | (84) |

A moderate dose response for duration of sleep parameter was observed for CBN. It can also be seen that THC at high doses (above 5 mg/kg) in fact increases the sleep latency (time required for falling asleep) and decreases the sleep duration. THC at medium concentrations (2-5 mg/kg) increases the onset of sleep. THC assists in awakening, mice administered with THC were much more vivid after awakening than mice of received diazepam that remained dizzy for a prolonged period of time. CBD assisted in sleeping already at 1 mg/kg dose (that correspond to 0.1 mg/kg in human). Typically, the conversion ratio from mice to human is 1 to 0.1 for cannabinoids (1 mg/kg in mice =0.1 mg/kg human).

TABLE 15
Cycle 2 - Effect of combinations of different
cannabinoids on sleep latency and duration

| | | | | Sleep latency | | Sleep duration | |
|---|---|---|---|---|---|---|---|
| Group | | | Dose | median | | median | |
| # | Test item | | mg/kg | (mean) | | (mean) | |
| 15F | Diazepam^{#} | | 2 | 4 | (4.3) | 56 | (51.7) |
| 16F | Vehicle | | — | 6.5 | (8.7) | 10.5 | (7.7) |
| 17F | CBN + | CBN | 0.02 | 7 | (7.2) | 9 | (9) |
| | CBD | CBD | 0.2 | | | | |
| 18F | | CBN | 0.1 | 7 | (7.2) | 5.5 | (5.2) |
| | | CBD | 1 | | | | |
| 19F | | CBN | 0.5 | 6.5 | (7.3) | 12 | (22) |
| | | CBD | 5 | | | | |
| 20F | | CBN | 2 | 6.5 | (6.5) | 54 | (64.5) |
| | | CBD | 20 | | | | |
| 21F | CBN + | CBN | 0.02 | 4 | (4) | 12 | (17.8) |
| | THC | THC | 0.5 | | | | |
| 22F | | CBN | 0.1 | 5 | (4.8) | 7 | (8.5) |
| | | THC | 2 | | | | |
| 23F | | CBN | 0.5 | 5.5 | (5.5) | 24.5 | (23.5) |
| | | THC | 5 | | | | |
| 24F | CBN + | CBN | 0.1 | 5.5 | (5.2) | 11 | (15.8) |
| | CBD + | CBD | 1 | | | | |
| | THC | THC | 2 | | | | |
| 25F | | CBN | 2 | 6 | (6) | 62.5 | (58) |
| | | CBD | 20 | | | | |
| | | THC | 0.5 | | | | |

Composition comprising CBN 2 mg/kg and CBD 20 mg/kg showed additive effect which increased upon addition of THC 0.5 mg/kg which at this concentration should not have had any effect.

TABLE 16
Cycle 3 - Effect of different terpenes on sleep latency and duration

| | | | | Sleep latency | | Sleep duration | |
|---|---|---|---|---|---|---|---|
| Group | | | | median | | median | |
| # | Test item | | Dose | (mean) | | (mean) | |
| 29F | Diazepam^{#} | | 2 | 2 | (2.3) | 60 | (66.3) |
| 30F | Vehicle | | — | 5 | (5.3) | 9 | (8.7) |
| 31F | Myrcene | | 20 | 5 | (4.7) | 9 | (8.7) |
| 32F | | | 100 | 5 | (5) | 7 | (7.3) |
| 33F | | | 300 | 3 | (3) | 31 | (61.3) |
| 34F | Linalool | | 20 | 5 | (5) | 6 | (5.3) |
| 35F | | | 100 | 5 | (5.5) | 6 | (5.3) |
| 36F | | | 300 | 2 | (2) | 120 | (97.3) |
| 37F | Myrcene + | Myrcene | 10 | 4 | (4) | 6 | (5.3) |
| | Linalool | Linalool | 10 | | | | |
| 38F | | Myrcene | 50 | 4 | (3.7) | 14 | (13.7) |
| | | Linalool | 50 | | | | |
| 39F | | Myrcene | 150 | 3 | (3) | 53 | (43) |
| | | Linalool | 150 | | | | |

| 40F | Valerian | 5 | 5 | (4.3) | 26 | (21.7) |
|---|---|---|---|---|---|---|
| 41F | | 20 | 5 | (5.3) | 27 | (23.7) |
| 42F | | 50 | 5 | (5.7) | 6 | (18.7) |

Very high concentration of terpenes induced sleeping and provided long sleep duration.

TABLE 17
Cycle 4 - Effect of different combinations of cannabinoids
and terpenes on sleep latency and duration

| | | | | Sleep latency | | Sleep duration | |
|---|---|---|---|---|---|---|---|
| Group | | | Dose | median | | median | |
| # | Test item | | mg/kg | (mean) | | (mean) | |
| 43F | Diazepam^{#} | | 2 | 3 | (3.3) | 69 | (76) |
| 44F | Vehicle | | — | 20 | (17.7) | 12 | (9.7) |
| 45F | CBN + | CBN | 0.1 | 6 | (7.3) | 6 | (7) |
| | CBD + | CBD | 1 | | | | |
| | β- | Myrcene | 20 | | | | |
| | myrcene | | | | | | |
| 46F | | CBN | 0.1 | 4 | (3.7) | 47 | (46) |
| | | CBD | 1 | | | | |
| | | Myrcene | 100 | | | | |
| 47F | | CBN | 0.5 | 4 | (4.7) | 21 | (17.3) |
| | | CBD | 1 | | | | |
| | | Myrcene | 20 | | | | |
| 48F | | CBN | 0.5 | 3 | (3.3) | 33 | (36.3) |
| | | CBD | 1 | | | | |
| | | Myrcene | 50 | | | | |
| 49F | | CBN | 0.5 | 4 | (4.7) | 72 | (71) |
| | | CBD | 1 | | | | |
| | | Myrcene | 100 | | | | |
| 50F | CBN + | CBN | 0.5 | 6 | (6) | 19 | (17.3) |
| | CBD + | CBD | 1 | | | | |
| | Linalool | Linalool | 20 | | | | |
| 51F | | CBN | 0.5 | 3 | (4.7) | 25 | (23.7) |
| | | CBD | 1 | | | | |
| | | Linalool | 50 | | | | |
| 52 F | | CBN | 0.5 | 4 | (3.7) | 68 | (68.3) |
| | | CBD | 1 | | | | |
| | | Linalool | 100 | | | | |
| 53F | CBN + | CBN | 0.5 | 2 | (2.3) | 63 | (76.7) |
| | CBD + | CBD | 1 | | | | |
| | THC + | THC | 1 | | | | |
| | Myrcene | Myrcene | 100 | | | | |
| 54F | CBN + | CBN | 0.5 | 4 | (3.7) | 55 | (58.3) |
| | CBD + | CBD | 1 | | | | |
| | THC + | THC | 1 | | | | |
| | Linalool | Linalool | 100 | | | | |

Interestingly, both tested terpenes: β-myrcene and linalool showed an evident dose response in combination with CBN and CBD. Such dose response was not seen when the terpenes were administered alone. Addition of THC to the combination at concentration of 0.5 mg/kg reduced the observed effect.

TABLE 18
Cycle 5 - Effect of different combinations with
high content of cannabinoids and low content
of terpenes on sleep latency and duration.

| | | | Sleep latency | | Sleep duration | |
|---|---|---|---|---|---|---|
| | | Dose | median | | median | |
| Group | Test Item | mg/kg | (mean) | | (mean) | |
| 55F | CBN | 100 | 4 | (4) | 85.5 | (85) |
| | CBD | 20 | | | | |
| 56F | CBN | 100 | 3 | (3) | 98 | (98) |
| | CBD | 20 | | | | |
| | THC | 20 | | | | |
| 57F | myrcene | 10 | 5 | (5) | 5 | (5) |
| 58F | linalool | 10 | 8 | (8) | 6.5 | (6) |
| 59F | CBN | 100 | 3 | (3) | 101.5 | (101) |
| | CBD | 20 | | | | |
| | Myrcene | 10 | | | | |
| 60F | CBN | 100 | 3 | (3) | 103 | (94) |
| | CBD | 20 | | | | |
| | Linalool | 10 | | | | |
| 61F | CBN | 100 | 5.5 | (6) | 112.5 | (106) |
| | CBD | 20 | | | | |
| | THC | 10 | | | | |
| | Myrcene | 10 | | | | |
| 62F | CBN | 100 | 6 | (6) | 114.5 | (113) |
| | CBD | 20 | | | | |
| | THC | 10 | | | | |
| | Linalool | 10 | | | | |

At the next step, we have compared the medians of a duration of sleep shown for different combinations and the calculated expected medians of duration of sleep for these combinations. The expected median of duration of sleep was calculated as follows: (observed median for cannabinoids combination−average median for negative control (NC))+(observed median for a terpene−NC). For the comparison, the median of negative control was subtracted from the medians observed for the tested combinations of cannabinoids and terpenes, i.e. (observed median for combination of cannabinoids and terpenes−NC). In case a median for a combination of cannabinoids with particular concentrations was not available, the median of the combination with higher concentration was used for calculation of the expected values. The results are presented in Table 19.

TABLE 19
Synergic sleep inducing effect of several composition
comprising cannabinoids and terpenes

| | Expected effect | Shown Effect | Synergy |
|---|---|---|---|
| 45F | 0.33 | 0 | No |
| 46F | 0 | 38.3 | YES |
| 47F | 3.66 | 12.3 | YES |
| 48F | 3.33 | 24.3 | YES |
| 49F | 3.33 | 63.3 | YES |
| 50F | 3.33 | 10.3 | Weak |
| 51F | 7.66 | 16.3 | Weak |
| 52F | 7.66 | 59.3 | YES |
| 53F | 2.33 | 54.3 | YES |
| 54F | 6.66 | 46.3 | YES |
| 59F | 76.83 | 92.8 | YES |
| 60F | 76.83 | 94.3 | YES |
| 61F | 89.33 | 103.8 | YES |
| 62F | 89.33 | 105.8 | YES |

It can be seen that all composition except for 45F provided a synergic effect on increasing sleep duration.

### Example 6. Viscosity of the Compositions

The objective of the present study was to measure the temperature upon which the composition of the present invention fluidizes and the viscosity value at that temperature.

Compositions A1 and A4, (Table 6) and compositions B1, B2, B3 and B4 (Table 8) were heated gradually at about 2° C./minute and their fluidity was evaluated by turning the vessel 90 degrees and monitoring the flow of the e-liquid (composition). Flow was observed when the sample compositions reached about 60° C.

A Brookfield viscometer DV-II with spindle number LV-3 was used to measure the viscosity of compositions at 60° C. The samples were heated to 60° C. by a double jacked glass tube and the viscosity was tested. As follows from these experiments, the viscosity of samples compositions was as follow A1=354 cP, A4=148 cP, B1=781 cP, B2=139 cP, B3=427 cP and B4=533 cP.

Although the present invention has been described herein above by way of particular embodiments thereof, it can be modified, without departing from the spirit and nature of the subject invention as defined in the appended claims.

1-46. (canceled)

47. A vaporizable composition, wherein the composition comprises from about 1 wt % to about 30 wt % of a cannabinoid cannabinol (CBN) and up to about 15 wt % of at least one terpene, and wherein the composition has the viscosity in the range of about 10 cP to about 1000 cP at 60° C.

48. The vaporizable composition of claim 47, wherein at least one of the following exists: (i) the composition comprises from about 3 wt % to about 25 wt %, or about 5 wt % to about 20 wt % of CBN; (ii) the composition comprises from about 2 wt % to about 12 wt % of terpenes; (iii) the viscosity of the composition is in the range of about 100 cP to about 700 cP at 60²C; (iv) the weight ratio of terpenes to cannabinoid(s) is in the range of 1:4 to 1:50; (v) the composition has an absorbance of less than 2 absorbance units (AU) or less than 1 AU at 293 nm, wherein the absorbance is measured upon 1:100 dilution of the composition in ethanol; (vi) the composition liquefies at a temperature between 50 to about 70° C.; (vii) the composition increases the duration of sleep and/or induces sleep; (viii) the composition is a vaporizable pharmaceutical composition.

49. The vaporizable composition of claim 47, wherein the at least one terpene is selected from the group consisting of β-myrcene, linalool, eucalaptol, limonene, α-pinene, citral, linalyl acetate, borneol, nerolidol, terpenolene, and any combination thereof.

50. The vaporizable composition of claim 47, wherein the composition further comprises at least one cannabinoid in addition to CBN.

51. The vaporizable composition of claim 50, comprising from about 80 wt % to about 95 wt % of cannabinoids.

52. The vaporizable composition of claim 50, wherein the at least one cannabinoid is selected from the group consisting of tetrahydrocannabinol (THC), cannabidiol (CBD), cannabigerol (CBG), tetrahydrocannabinolic acid, CBDA, and any combination thereof.

53. The vaporizable composition of claim 52, comprising THC, CBD or a combination thereof.

54. The vaporizable composition of claim 53, wherein at least one of the following exists: (i) the composition comprises no more than 30 wt %; (ii) the composition comprises from about 5 wt % to about 20 wt % of THC; or (iii) the composition comprises from about 45 wt % to about 85 wt % of CBD, the composition comprises CBD and THC and wherein the weight ratio of CBD to THC to is at least 3 to 2.

55. The vaporizable composition of claims 47, comprising from about 10 wt % to about 99 wt % of cannabis extract.

56. The vaporizable composition of claim 55, wherein the cannabis extract comprises from about 80 wt % to about 98 wt % of cannabinoids and from about 2 wt % to about 20 wt % of terpenes.

57. The vaporizable composition of claim 55, wherein the cannabis extract comprises at least one of (i) from about 20 wt % to about 85 wt % of cannabinoids and up to about 2 wt % of terpenes, or (ii) from about 5 wt % to about 25 wt % CBN and from about 2 wt % to about 12 wt % of terpenes selected from β-myrcene, linalool, eucalaptol, limonene, α-pinene, citral, linalyl acetate, borneol, nerolidol, terpenolene, and any combination thereof, or (iii) wherein the viscosity of the composition is in the range of about 100 cP to about 700 cP at 60° C. 58 (New) The vaporizable composition of claim 47, comprising (i) from about 3 to about 25 wt % CBN, from 50 to 85 wt % CBD and from 1 to 15 wt % of a terpene selected from β-myrcene, linalool and combination thereof or (ii) from about 5 to about 20 wt % CBN, from 45 to 75 wt % CBD and from 8 to 12 wt % of a terpene selected from linalool, β-myrcene and combination thereof, and optionally further comprising from about 5 to about 15 wt % THC.

59. The vaporizable composition of claim 58, wherein the composition has a synergic sleep inducing and/or sleep prolonging effect.

60. The vaporizable composition of claim 47, comprising only compounds naturally present in cannabis plants.

61. The vaporizable composition of claim 47, further comprising additives.

62. The vaporizable composition of claim 61, wherein the additive is selected from the group consisting of an oil, triglyceride, propylene glycol, butylene glycol, hexylene glycol, glycerin, polyethylene glycol, organic solvent and vitamin E.

63. A container comprising the vaporizable composition of claim 47.

64. A method for treating a sleep disorder in a subject in need thereof, said method comprising administering via vaporization the vaporizable composition of claim 47.

65. The method of claim 64, wherein the sleep disorder is selected from dyssomnia, insomnia, circadian rhythm sleep disorders, and parasomnia.

66. The method of claim 64, wherein the method comprises at least one of (i) using a vaporizer device, or (ii) administering from about 1 to about 120 mg/day of CBN.
